(19) United States
(12) Patent Application Publication
(10) Pub. No.: US 2025/0171768 A1
(43) Pub. Date: May 29, 2025

(54) METHOD AND APPARATUS FOR PROCESSING TISSUE SAMPLES

(71) Applicant: S2 Genomics, Inc., Livermore, CA (US)

(72) Inventors: John BASHKIN, Fremont, CA (US); Stevan Bogdan JOVANOVICH, Livermore, CA (US); Danielle MEYER, Livermore, CA (US); Nathan PEREIRA, Pleasanton, CA (US); Kaiwan CHEAR, Livermore, CA (US); David EBERHART, Santa Clara, CA (US)

(21) Appl. No.: 18/843,294

(22) PCT Filed: Mar. 2, 2023
(86) PCT No.: PCT/US2023/014338

§ 371 Date: Sep. 2, 2024

Related U.S. Application Data
(60) Provisional application No. 63/315,960, filed on Mar. 2, 2022.

(51) Int. Cl. C12N 15/10; B01L 3/00

(52) U.S. Cl. CPC C12N 15/1065; B01L 3/502761; B01L 2200/04; B01L 2200/0668; B01L 2200/10; B01L 2200/16

(57) ABSTRACT

A cartridge for dissociating tissue, comprising: a processing chamber comprising a stator, a side wall, a top orifice, and a first processing chamber port positioned in the side wall; and a grinder assembly comprising a plunger comprising a rotor, a grinder assembly slidably positioned in the processing chamber through the top orifice; wherein: the stator comprises a plurality of teeth arranged in a spaced-apart array of rings; and the rotor comprises one or more central teeth and a plurality of teeth arranged in a spaced-apart array of rings, wherein one ring of teeth is positioned at or substantially at a circumference of the rotor; wherein the rings in the stator and the rings in the rotor are positioned such that when the rotor contacts the stator, rings of teeth in the stator mesh with the one or more central teeth and rings of teeth in the rotor

### CROSS REFERENCE TO RELATED APPLICATIONS

This application claims the benefit of the priority date of U.S. Provisional application 63/315,960, filed Mar. 2, 2022, the contents of which are incorporated herein in their entirety.

### BACKGROUND OF THE INVENTION

### Field of Invention

This invention relates to the field of sample preparation from biological materials. More specifically, the invention relates to the processing of tissues into single cells, nuclei or subcellular organelles, such as mitochondria and ribosomes, for bioanalysis. This invention further relates to a system comprising a cartridge comprising a tissue processing chamber including a stator and a grinding element comprising a rotor. The stator can comprise teeth that mesh with teeth on a bottom surface of the rotor, that is, the teeth in one part are staggered with respect to the teeth on the other part.

### Background

Single-cell and single-nucleus sequencing is rapidly changing the state of knowledge of cells and tissue, discovering new cell types, and increasing the understanding of the diversity of how cells and tissue function. Single-cell RNA sequencing is being applied to development, brain structure and function, tumor progression and resistance, immunogenetics, and more (Shapiro E. Biezuner T, Linnarsson S. Single-cell sequencing-based technologies will revolutionize whole-organism science. Nat Rev Genet. 2013;14(9):618-30. PMID: 23897237). Single cell or nuclei sequencing has highlighted the complexity of cellular expression, and the large heterogeneity from cell-to-cell, and from cell type-to-cell type (Buettner F. Natarajan K N, Casale F P, Proserpio V, Scialdone A, Theis F J, Teichmann S A, Marioni J C, Stegle O. Computational analysis of cell-to-cell heterogeneity in single-cell RNA-sequencing data reveals hidden subpopulations of cells. Nat Biotechnol. 2015; 33(2):155-60. PMID: 25599176) (Wang., Y. and N. E. Navin. Advanced and Applications of single-cell sequencing technologies. Molecular Cell. 2015. 58:598-609. PMID 26000845.).

Single cell and single nuclei sequencing technology and methods using NGS are rapidly evolving. Common components are incorporation of a marker or barcode for each cell and molecule, reverse transcriptase for RNA sequencing, amplification, and pooling of sample for NGS and NNGS (collectively termed NGS) library preparation and analysis. Starting with isolated single cells in wells, barcodes for individual cells and molecules have been incorporated by reverse transcriptase template switching before pooling and polymerase chain reaction (PCR) amplification (Islam S. et. al. Genome Res. 2011; 21(7):1160-7.) (Ramsköld D. et. al. Nat Biotechnol. 2012; 30(8):777-82.) or on a barcoded poly-T primer with linear amplification (Hashimshony T. et. al. Cell Rep. 2012 Sep. 27;2(3):666-73.) and unique molecular identifiers (Jaitin D. A. et. al. Science. 2014; 343(6172):776-9.).

Standarization is necessary before routine single-cell preparation can be performed, particularly in clinical settings. In addition, the length of the process and the process of dissociation can lead to the tissue and cells changing physiology such as altering their expression of RNA and proteins in response to the stresses of the procedure, accentuated by potentially long processing times.

The direct dissociation of tissue into nuclei avoids many of these issues and single nuclei RNA sequencing (snRNA-Seq) can give a snapshot of gene expression (Habib N, Li Y, Heidenreich M, Swiech L, Avraham-Davidi I, Trombetta J J, Hession C, Zhang F, Regev A. Div-Seq: Single-nucleus RNA-Seq reveals dynamics of rare adult newborn neurons. Science. 2016 Aug. 26;353(6302):925-8. doi: 10.1126/science.aad7038. Epub 2016 Jul. 28.; Grindberg RV, Yee-Greenbaum J L, McConnell M J, Novotny M, O'Shaughnessy A L, Lambert G M, Arauzo-Bravo M J, Lee J, Fishman M, Robbins GE, Lin X, Venepally P, Badger J H, Galbraith D W, Gage FH, Lasken R S. RNA-sequencing from single nuclei. Proc Natl Acad Sci U S A. 2013 Dec. 3;110(49):19802-7. doi: 10.1073/pnas. 1319700110. Epub 2013 Nov. 18.).

The production of nuclei from tissue can be performed using a Dounce homogenizer in the presence of a buffer with a detergent that lyses cells but not nuclei. Nuclei can also be prepared starting from single cell suspensions by addition of a lysis buffer such as 10 mM Tris-HCl, 10 mM NaCl, 3 mM MgCl2 and 0.005% Nonidet P40 in nuclease-free water and incubation for 5 min on ice before centifugation to pellet the nuclei followed by resuspension in a resuspension buffer such as 1X PBS with 1.0% BSA and 0.2 U/μl RNase Inhibitor. The nuclei may be repeatedly pelleted and resuspended to purify them or density gradients or other purification methods used. The titer and viability of the nuclei suspension is usually determined using optical imaging with a microscope and haemocytometer, or an automated instrument with viability determined using Trypan blue or fluorescent dyes.

### BRIEF SUMMARY OF THE INVENTION

In one embodiment a Sample processing System is used for tissue processing. A Tissue Processing System embodiment can be implemented as a flexible, extensible system that can process solid or liquid tissue and other samples into single cells, nuclei, organelles, and biomolecules with mechanical and enzymatic or chemical processes to produce single nuclei, subcellular components, and biomolecules such as macromolecules comprised of nucleic acids, comprised of DNA and RNA; proteins; carbohydrates; lipids; biomolecules with multiple types of macromolecules, metabolites; and other biological components, including natural products for bioanalysis. In some embodiments, the Tissue Processing System performs affinity or other purifications to enrich or deplete cell types, organelles such as nuclei, mitochondria, ribosomes, or other organelles, or extracellular fluids. In some embodiments the Tissue Processing System can perform NGS library preparation. In some embodiments, the Tissue Processing System processes tissue into single-nuclei libraries for sequencing including Sanger, NGS, single nuclei NGS, and other nucleic acid sequencing technologies, or protoeomics, or other analytical methods.

The Sample processing System can have multiple subsystems and modules that perform processing or analysis. In an embodiment of the Sample processing System, one or more cartridges performs one or more steps in the processing workflow. In some embodiments the cartridges have multiple processing sites such as processing chambers that can process more than one sample. In some embodiments a cap couples mechanical disruption on the cartridge from a Physical Dissociation Subsystem. In some embodiments reagents from an Enzymatic and Chemical Dissociation Subsystem are delivered to the cartridge by a Fluidic Subystem to regions that are used as processing Chambers and Post-Processing Chambers to disrupt or dissociate specimen and process the cells, subcellular components, and biomolecules for bioanalysis.

The addition of fluids can be controlled by a Fluidic Subsystem with the complete system controlled by software in a Control Subsystem which can include the user interface through a device comprised of monitor, embedded display, touch screen; or through audio commands through the system or an accessory devices such as a cell phone or microphone. In some instances the Control Subsystem can include interfaces to laboratory information management systems, other instruments, databases, analysis software, email, and other applications.

In some embodiments, the amount of dissociation is monitored at intervals during the dissociation and in some instances the yield is determined during or after processing using a Measurement Subsystem. The degree of dissociation can be determined inside the main dissociation compartment and/or in a separate compartment or channel, and/or in the external instrument.

In some embodiments, cell or organelle or other imaging or labeling solutions, such as cell type specific antibodies, stains, or other reagents, can be added to the tissue or single cells or nuclei before, during, or after processing. The imaging can capture cells, subcellular structures, cell health assays of apoptosis, necrosis, or cytoxicity, or histological or other data. In some embodiments the images can be analyzed to direct the operation and workflow of the Sample processing System through decisions trees, hash tables, machine learning, or artificial intelligence. In other embodiments the imaging or labeling solutions can contain DNA or other barcodes.

In some embodiments, single cells or nuclei in suspension or on surfaces are further processed using magnetic bead or particle technologies using a Magnetic processing module to purify or deplete cell types, nuclei, nucleic acids, or other biomolecules.

The term singulated cells is used to mean single cells in suspension or on a surface or in a well including a microwell or nanowell such that they can be processed as single cells. The term singulated cells is also used at times to encompass single nuclei. The term nuclei suspension is also used at times to encompass single cell suspensions. Similarly, singulated nuclei and singulated organelles refer to single nuclei or organelles in suspension or on a surface or in a well including a microwell or nanowell such that they can be processed as single entities.

In one embodiment, the specimen is added to a cartridge which performs both physical and enzymatic dissociation of the tissue. In some embodiments the Tissue Processing System performs tituration and other physical dissociation modalities as a step or steps in the process of singulating cells. The physical dissociation modalities include passing the specimen through screens, filters, orifices, grinding, blending, sonication, smearing, bead beating, and other methods known to one skilled in the art to physically disrupt tissue to help produce single cells or nuclei or nucleic acids or other biomolecules.

In some embodiments, the Sample processing System, such as a Tissue Processing System embodiment, uses enzymes to assist in the process of singulating cells or nuclei including enzymes to preserve nucleic acids and prevent clumping. The enzymes are comprised of, but not limited to, collagenases (e.g., collagenases type I, II, III, IV, and others), elastase, trypsin, papain, tyrpLE, hyaluronidase, chymotrypsin, neutral protease, pronase, liberase, clostripain, caseinase, neutral protease (Dispase®), DNAse, protease XIV, RNase inhibitors, or other enzymes, biochemicals, or chemicals such as Triton X-100, Nonidet P40, detergents, surfactants, etc. In other embodiments, different reagents or mixtures of reagents are applied sequentially to dissociate deparaffinized, rehydrated formalin-fixed, paraffin embedded (FFPE) specimens 150 into single-cell or single nuclei suspensions. In other embodiments, reagents containing detergents or surfactants are applied to dissociate deparaffinized, rehydrated FFPE specimens into single nuclei suspensions.

In some embodiments the Tissue Processing System produces suspensions of known titers. In some embodiments the Tissue Processing System monitors the amount of singulation of a sample and adjusts the treatment time and concentration of enzymes, chemicals, mechanical disruption, or other dissociation agents by monitoring of the dissociation, for example by the production of single cells or nuclei. The monitoring can be in real time, in intervals, or endpoints or any combinations thereof.

The Tissue Processing System can in some embodiments select from sets of reagents to deparaffinize, rehydrate, reverse crosslinks, and dissociate tissue by adjusting the production of single nuclei by monitoring by the system, in some instances in real time, at intervals, or as an endpoint the titer, quality, or other attributes of the single nuclei suspensions.

The Tissue Processing System has advantages over existing technology and can produce single nuclei, mitochondria, or biomolecules from tissue in an automated and standardized instrument that can in some embodiments process the specimens into NGS libraries or other preparations. The Tissue Processing System will enable users, e.g., researchers, clinicians, forensic scientists, and many disciplines to perform identical processing on biosamples, reducing user variability, and throughput constraints of manual processing.

Embodiments of the Tissue Processing System can prepare single nuclei suspensions or single cells or nucleic acids for analysis by methods comprised of bulk and single nuclei DNA sequencing, DNA microarrays, RNA sequencing, mass spectrometry, Raman spectroscopy, electrophysiology, flow cytometry, mass cytometry, and many other analytical methods well known to one skilled in the art including multidimensional analysis (e.g., LC/MS, CE/MS, etc.) and multi-'omics (e.g., genomic and proteomic analysis, genomic and cell surface analysis, etc.).

The Tissue Processing System embodiment described is compatible with commercially available downstream library preparation and analysis by NGS sequencers. The term NGS is used to connote either NGS or nanopore or single molecule sequencing or other sequencing methods or sample preparation methods as appropriate without limitation. As contemplated herein, next generation sequencing refers to high-throughput sequencing, such as massivley parallel sequencing (e.g., simultaneously (or in rapid succession) sequencing any of at least 1,000, 100,000, 1 million, 10 million, 100 million, or 1 billion polynucleotide molecules). Sequencing methods may include, but are not limited to: high-throughput sequencing, pyrosequencing, sequencing-by-synthesis, single-molecule sequencing, nanopore sequencing, semiconductor sequencing, sequencing-by-ligation, sequencing-by-hybridization, massively parallel signature sequencing (MPSS), Polony sequencing, DNA cluster sequencing (e.g., Illumina), RNA-Seq (e.g., Illumina), Digital Gene Expression (e.g., Helicos), next generation sequencing, Single Molecule Sequencing by Synthesis (SMSS) (e.g., Helicos), massively-parallel sequencing, Clonal Single Molecule Array (e.g., Solexa), shotgun sequencing, Maxam-Gilbert or Sanger sequencing, primer walking, sequencing using single molecule real time (SMRT) sequencing (e.g., PacBio), SOLID, Ion Torrent semiconductor sequencing, Genius (e.g., GenapSys) or nanopore (e.g., Oxford Nanopore, Roche) platforms DNA nanoball sequencing (e.g., Complete Genomics), and any other sequencing methods known in the art.

In another aspect provided herein is an apparatus, composition of matter, or article of manufacture, and any improvements, enhancements, and modifications thereto, as described in part or in full herein and as shown in any applicable Figures, including one or more features in one or more embodiment.

In another aspect provided herein is an apparatus, composition of matter, or article of manufacture, and any improvements, enhancements, and modifications thereto, as described in part of in full herein and as shown in any applicable Figures, including each and every feature.

In another aspect provided herein is a method or process of operation or production, and any improvements, enhancements, and modifications thereto, as described in part or in full herein and as shown in any applicable Figures, including one or more feature in one or more embodiment.

In another aspect provided herein is a method or process of operation or production, and any improvements, enhancements, and modifications thereto, as described in part or in full herein and as shown in any applicable Figures, including each and every feature.

In another aspect provided herein is a product, composition of matter, or article of manufacture, and any improvements, enhancements, and modifications thereto, produced or resulting from any processes described in full or in part herein and as shown in any applicable Figures.

In one embodiment the single-cell or nuclei suspension is prepared for a bioanalysis module for downstream analysis including but not limited to sequencing, next generation sequencing, proteomic, genomic, gene expression, gene mapping, carbohydrate characterization and profiling, lipid characterization and profiling, flow cytometry, imaging, DNA or RNA microarray analysis, metabolic profiling, functional, or mass spectrometry, or combinations thereof.

In another aspect provided herein is a data analysis system that correlates, analyzes, and visualizes the analytical information of a sample component such as its degree of single cell or nuclei dissociation, with the processing step and measures the change over time, and/or amount of enyzmatic activity, and/or physical and/or chemical or enzymatic disruptions of the original biological specimen.

In another aspect provided herein is a data analysis system that correlates, analyzes, and visualizes the analytical information of a sample component such as its degree of single cell or nuclei dissociation, with the processing step and measures the change over time, and/or amount of enyzmatic/chemical activity, and/or physical disruptions of the original biological specimen and adjusts the processing parameters from the analytical information.

In another aspect, provided herein is a system comprising: (a) an instrument comprising: (i) one or more cartridge interfaces configured to engage a cartridge; (ii) a fluidics module comprising: (1) one or more containers containing one or more liquids and/or gasses and/or solids that may be dissolved to form liquids; (2) one or more fluid lines connecting the containers with fluid ports in the cartridge interface; and (3) one or more pumps configured to move liquids and/or gasses into and/or out of the fluid port(s); (iii) a mechanical module comprising an actuator; (iv) optionally, a magnetic processing module comprising a source of magnetic force, wherein the magnetic force is positioned to form a magnetic field in the processing chamber; (v) optionally, a measurement module; (vi) optionally, a control module comprising a processor and memory, wherein the memory comprises code that, when executed by the processor, operates the system; and (b) one or more cartridges, each engaged with one of the cartridge interfaces, wherein each cartridge comprises: (i) a sample inlet port; (ii) one or more cartridge ports communicating with the fluid ports in the cartridge interface; (iii) a processing chamber communicating with the sample inlet port and with at least one cartridge port, and comprising a tissue disruptor (also referred to herein as a “grinder assembly”) configured for mechanical disruption of tissue, wherein the tissue disruptor engages with and is actuated by the actuator when the cartridge is engaged with the cartridge interface; (iv) an optional strain chamber communicating with the processing chamber configured to separate cells and/or nuclei from disrupted tissue; (v) an optional post-processing chamber communicating with the strain chamber, optionally communicating with one or more cartridge ports and configured to perform one or more processing steps on separated cells, nuclei or mitochondria when required; and (vi) optionally, one or more waste chambers fluidically connected with the processing chamber. In one embodiment the tissue disruptor comprises a grinder, a pestle or a variable orifice. In another embodiment the system further comprises a barcode reader. In another embodiment the system comprises a measurement module (vii) that performs optical imaging to measure titer, clumping, and/or viability of cells or nuclei or properties of biomolecules. In another embodiment the system comprises a measurement module (viii) and a control system (ix), wherein the measurement module measures, and one or more time points, characteristics of a sample in the processing chamber, and control system comprises code that determines a state of the sample, e.g., viability or degree of single cell or nuclei dissociation or degree of deparaffinization or rehydration, etc., and optionally adjusts processing parameters. In another embodiment the system further comprises (c) a device to hold one or more FFPE tissues during the cartridge processing. In another embodiment the system further comprises (d) an analysis module, wherein an input port of the analysis module is in fluid communication with the processing chamber. In another embodiment the analysis module performs an analysis selected from one or more of: DNA sequencing, next generation DNA sequencing, proteomic analysis, genomic analysis, gene expression analysis, gene mapping, carbohydrate characterization and profiling, lipid characterization and profiling, flow cytometry, imaging, DNA or RNA microarray analysis, metabolic profiling, functional analysis, and mass spectrometry. In another embodiment the cartridge interface comprises a means of positioning the cartridge in the instrument that engages the fluidic module and the mechanical module and optionally is temperature controlled. In another embodiment the cartridge is disposable.

In another aspect provided herein is a cartridge comprising: (i) a sample inlet port; (ii) one or more cartridge ports configured to communicate with fluid ports in a cartridge interface; (iii) a processing chamber communicating with the sample inlet port and with at least one cartridge port, and comprising a tissue disruptor configured for mechanical disruption of tissue, wherein the tissue disruptor engages with and is actuated by the actuator when the cartridge is engaged with the cartridge interface; (iv) a post-processing chamber containing one or more strainers, optionally communicating with one or more cartridge ports and configured to perform one or more processing steps on separated cells; and (v) optionally, one or more waste chambers fluidically connected with the post-processing chamber. In another embodiment the cartridge further comprises a cap that opens and closes the sample inlet port. In another embodiment the cap comprises a tissue disruptor element that moves about rotationally and back and forth along an axis. In another embodiment the cartridge further comprises a grinding element for grinding tissue in the processing chamber. In another embodiment the cartridge further comprises a barcode comprising information about the cartridge and/or its use. In another embodiment the cartridge further comprises a plunger configured to move slideably within the processing chamber.

In another aspect provided herein is a cartridge for dissociating tissue, comprising: a processing chamber comprising a stator, a side wall, a top orifice, and a first processing chamber port positioned in the side wall; and a grinder assembly comprising a plunger comprising a rotor, the grinder assembly slideably positioned in the processing chamber through the top orifice; wherein: the stator comprises a plurality of teeth arranged in a spaced-apart array of rings; and the rotor comprises one or more central teeth and a plurality of teeth arranged in a spaced-apart array of rings, wherein one ring of teeth is positioned at or substantially at a circumference of the rotor; wherein the rings in the stator and the rings in the rotor are positioned such that when the rotor contacts the stator, the rings of teeth in the stator mesh with the one or more central teeth and rings of teeth in the rotor.

### BRIEF DESCRIPTION OF THE DRAWINGS

The skilled artisan will understand that the drawings, described below, are for illustration purposes only. The drawings are not intended to limit the scope of the present teachings in any way.

FIG. 1 shows a Sample processing System that processes specimens into biocomponents such as single cells or nuclei for bioanalysis.

FIG. 2 shows an overview of a Tissue Processing System and some exemplary modules. Tissue specimens or other specimens are processed into single cells, nuclei, nucleic acids, single-cell libraries, and other biologicals through the use of one or more cartridges and one or more of the Physical Dissociation Subsystem, the Enzymatic and Chemical Dissociation Subsystem, the Measurement Subsystem, the Fluidic Subsystem, the Control Subsystem, and a Magnetic Module.

FIG. 3 shows the overall design concept for a prototype showing functional system and a few example modalities of mechanical disruption and examples of chemicals and enyzmes to dissociate FFPE tissue specimens into single cells, nuclei, and other biomolecules.

FIG. 4 shows an example of a Single-Sample Tissue Processing System with mechanical disruption in a single cartridge with a bank of enzymes and reagents located in the instrument to dissociate solid tissue specimens into single cells, nuclei, and other biomolecules.

FIG. 5 shows another example of a Single-Sample Tissue Processing System with mechanical disruption in a single cartridge with a bank of enzymes and reagents located separately from the instrument in a reagent module.

FIG. 6 shows the front of an example of a Single-Sample Tissue Processing System to dissociate tissue specimens into single nuclei suspensions, and other biomolecules using a cartridge.

FIG. 7 shows the back of an example of the Single-Sample Tissue Processing System.

FIG. 8A-C shows an example of a cartridge with processing, post-processing, and vacuum trap chambers for processing tissue specimens into single nuclei, single cells, and other biomolecules.

FIG. 9A-D show an example of adding reagents to a cartridge with a tissue ring, mixing the reagents, removing the reagents, and mechanically disrupting the tissue in the tissue ring for processing solid tissue specimens into single cells, nuclei, and other biomolecules and details of the assembly of the cap.

FIG. 10 shows an exemplary computer system.

FIG. 11 shows a cartridge architecture using pinch valves to direct liquid flows.

FIG. 12 shows an exemplary cartridge fluidic architecture.

FIG. 13 shows a cutaway view of an exemplary cartridge.

FIG. 14 shows a top-down view of an exemplary cartridge.

FIG. 15 shows an exemplary grinder assembly.

FIG. 16 shows a cutaway view of an exemplary cartridge with a grinder assembly positioned for insertion into the processing chamber.

FIG. 17 shows an exemplary cartridge with a grinder assembly inserted into the processing chamber.

FIGS. 18A-B show an exemplary cartridge with a feature designed to center the head of the rotor in the processing chamber and set the bottom gap and side gaps between the rotor and the wall of the processing chamber.

FIGS. 19A-D show a port cover with a low durometer over a port secured by a port cover retaining cylinder, or a crimp, or a heat staked port cover retaining cylinder.

FIGS. 20A-E show a cap engaging with a rotary motor adapter and with a cartridge with a processing postprocessing and vacuum trap chambers for processing solid tissue specimens into single cells, nuclei, and other organelles or biomolecules.

FIG. 21 shows an exemplary workflow to deparaffinize and rehydrate FFPE specimens followed by nuclei isolation.

FIG. 22A shows nuclei from a manually deparaffinized and rehydrated FFPE specimen and FIG. 22 B shows nuclei from an automated deparaffinized and rehydrated FFPE specimen.

FIG. 23A-C shows a TapeStation size separation with the expected peak for a Flex kit from the manufacturer (A), the separation from a manually deparaffinized and rehydrated FFPE specimen with automated dissociation into nuclei (B), and an automatically deparaffinized and rehydrated FFPE specimen with automated dissociation into nuclei (C).

FIG. 24A shows nuclei from an automatically deparaffinized and rehydrated FFPE specimen that was then automatically processed in a cartridge into nuclei and FIG. 24 B shows an electropherogram of RNA extracted from the nuclei.

### DETAILED DESCRIPTION OF THE INVENTION

NGS, mass spectrometry, fluorescent activated cell sorting (FACS), and other modern high-throughput analysis systems have revolutionized life and medical sciences. The progression of information has been from the gross level of organism, to tissue, and now to single cell analysis. Single cell analysis of genomic, proteomic including protein expression, carbohydrate, lipid, and metabolism of individual cells is providing fundamental scientific knowledge and revolutionizing research and clinical capabilities.

All patents, patent applications, published applications, treatises and other publications referred to herein, both supra and infra, are incorporated by reference in their entirety. If a definition and/or description is set forth herein that is contrary to or otherwise inconsistent with any definition set forth in the patents, patent applications, published applications, and other publications that are herein incorporated by reference, the definition and/or description set forth herein prevails over the definition that is incorporated by reference.

Specimen: The term “specimen,” as used herein, refers to an in vitro cell, cell culture, virus, bacterial cell, fungal cell, plant cell, bodily sample, FFPE sample, or tissue sample that contains genetic material. In certain embodiments, the genetic material of the specimen comprises RNA. In other embodiments, the genetic material of the specimen is DNA, or both RNA and DNA. In certain embodiments the genetic material is modified. In certain embodiments, a tissue specimen includes a cell isolated from a subject. A subject includes any organism from which a specimen can be isolated. Non-limiting examples of organisms include prokaryotes, eukaryotes, or archaebacteria, including bacteria, fungi, animals, plants, or protists. The animal, for example, can be a mammal or a non-mammal. The mammal can be, for example, a rabbit, dog, pig, cow, horse, human, or a rodent such as a mouse or rat. In particular aspects, the tissue specimen is a human tissue sample. The tissue specimen can be liquid, for example, a blood sample, red blood cells, white blood cells, platelets, plasma, serum. The specimen, in other non-limiting embodiments, can be saliva, a cheek, throat, or nasal swab, a fine needle aspirate, a tissue print, cerebral spinal fluid, mucus, lymph, feces, urine, skin, spinal fluid, peritoneal fluid, lymphatic fluid, aqueous or vitreous humor, synovial fluid, tears, semen, seminal fluid, vaginal fluids, pulmonary effusion, serosal fluid, organs, bronchio-alveolar lavage, tumors, frozen cells, or constituents or components of in vitro cell cultures. In other aspects, the tissue specimen is a solid tissue sample or a frozen tissue sample or a biopsy sample such as a fine needle aspirate or a core biopsy or a resection or other clinical or veterinary specimen. In other aspects, the tissue specimen is a FFPE preserved sample such as a biopsy sample such as a fine needle aspirate or a core biopsy or a resection or other clinical or veterinary specimen. In still further aspects, the specimen comprises a virus, bacteria, or fungus. The specimen can be an ex vivo tissue or sample or a specimen obtained by laser capture microdissection. The specimen can be a fixed specimen, including as set forth by U.S. Published Patent Application No. 2003/0170617 filed Jan. 28, 2003.

In some embodiments, the single cells can be analyzed further for biomolecules including one or more polynucleotides or polypeptides or other macromolecules. In some embodiments, the polynucleotides can include a single-stranded or double-stranded polynucleotide. In some embodiments, the polypeptide can include an enzyme, antigen, hormone or antibody. In some embodiments, the one or more biomolecules can include RNA, mRNA, cDNA, DNA, genomic DNA, microRNA, long noncoding RNA, ribosomal RNA, transfer RNA, chloroplast DNA, mitochondrial DNA, or other nucleic acids including modified nucleic acids and complexes of nucleic acids with proteins or other macromolecules.

It will be readily apparent to one of ordinary skill in the art that the embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

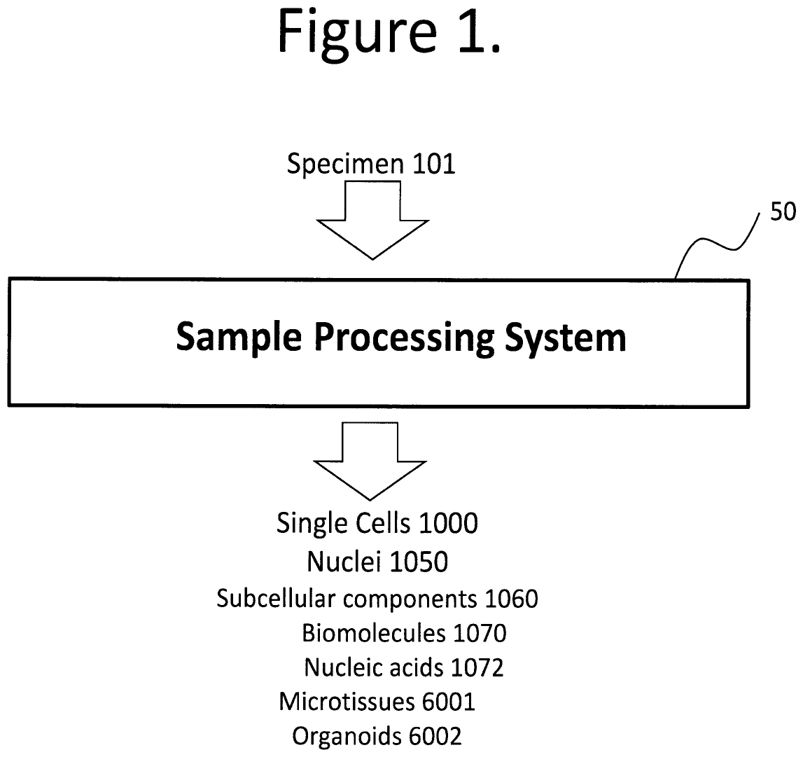

FIG. 1 shows a Sample processing System 50 that can input specimen 101 and process them to produce biologicals such as single cells 1000 or nuclei 1050, microtissues 6001, organoids 6002, or other biocomponents comprised of subcellular components 1060, and biomolecules 1070 such as macromolecules 1071 and nucleic acids 1072, comprised of DNA 1073 and RNA 1074; proteins 1075; carbohydrates 1076; lipids 1077; biomolecules 1070 with multiple types of macromolecules 1071; metabolites 1078; and other biological components, including natural products 1079 for bioanalysis.

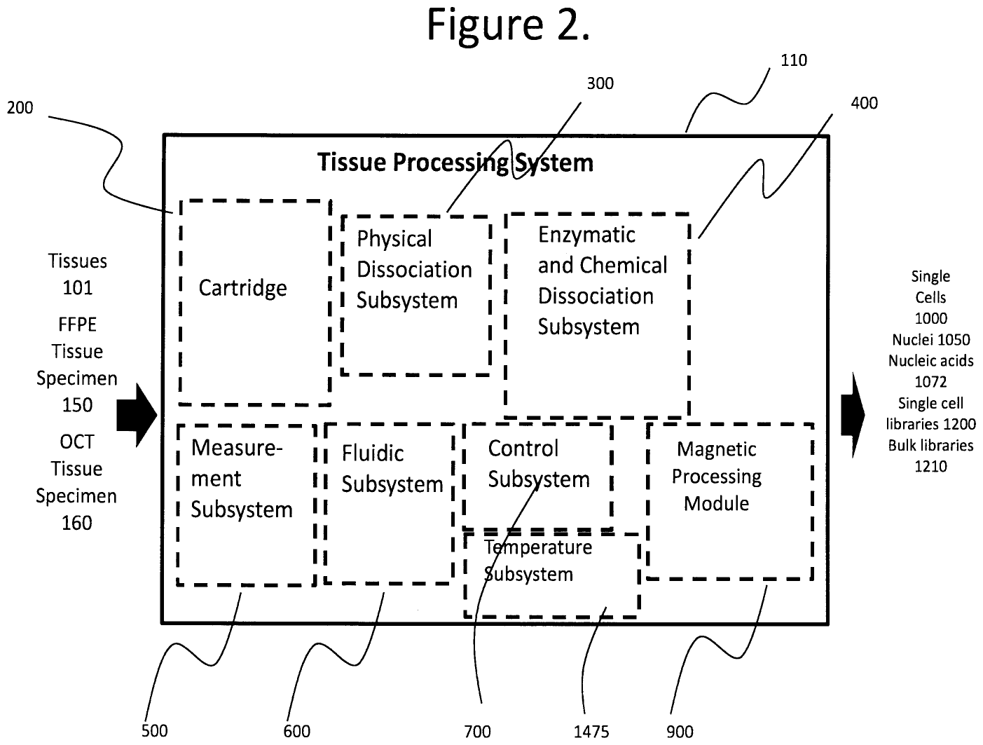

Referring to FIG. 2, in many embodiments, the Tissue Processing System 110 processing is performed in cartridges 200 in the system. Tissue samples, e.g., fresh tissue 101, FFPE tissue specimens 150 or OCT (optimal cutting temperature) tissue specimens 160 or other specimens 101 are converted to single nuclei 1050, single cells 1000, or other organelles, or biomolecules or single cell libraries 1200 or bulk libraries 1210 through the use of cartridge 200 with one or more of the Physical Dissociation Subsystem 300, the Enzymatic and Chemical Dissociation Subsystem 400, the Measurement Subsystem 500, the Fluidic Subsystem 600, the Control Subsystem 700, the Magnetic Module 900, and the Temperature Subsystem 1475.

The Physical Dissociation Subsystem 300 can perform mixing or perform physical disruption by passing the specimen through orifices, grinding, rotating a rotor with features to dissociate tissue, forcing tissue through screens or mesh, sonication, ultrasonics, blending, homogenization, bead beating, and other methods known to one skilled in the art to physically disrupt tissue to help produce single cells.

The Enzymatic and Chemical Dissociation Subsystem 400 can perform deparaffinization by adding xylene or xylene substitutes to the cartridge and perform rehydration by adding mixtures of ethanol with increasing amounts of water or buffer. The Enzymatic and Chemical Dissociation Subsystem 400 can perform crosslink reversal and/or enzymatic disruption by using heat or adding formulations of reagents or mixture of components comprised of but not limited to proteinase K, collagenases (e.g., collagenases type I, II, III, IV, and others), elastase, trypsin, papain, hyaluronidase, chymotrypsin, neutral protease, clostripain, caseinase, neutral protease (Dispase®), DNAse, protease XIV, RNase inhibitors, or other enzymes, biochemicals, or chemicals such as EDTA, protease inhibitors, buffers, acids, or base.

Another aspect or the Enzymatic and Chemical Dissociation Subsystem 400 can perform chemical disruption or chemical and enzymatic disruption is by adding formulations of chemicals that can disrupt tissue or cellular integrity such as Triton X-100, Tween, Nonident P40, octyl glucoside, polyoxyethylene(9)dodecyl ether, digitonin, IGEPAL™ CA630 octylphenyl polyethylene glycol, n-octyl-beta-Dglucopyranoside (betaOG), n-dodecyl-beta, Tween™ 20 polyethylene glycol sorbitan monolaurate, Tween™ 80 polyethylene glycol sorbitan monooleate, polidocanol, n-dodecyl beta-D-maltoside (DDM), NEMO nonylphenyl polyethylene glycol, C12E8 (octaethylene glycol ndodecyl monoether), hexaethyleneglycol mono-n-tetradecyl ether (C14E06), octyl-betathioglucopyranoside (octyl thioglucoside, OTG), Emulgen, and polyoxyethylene 10 lauryl etherother surfactants, or detergents or chemicals that can dissociate tissue into cells or produce nuclei or other organelles.

In other embodiments, different reagents or mixtures of reagents are applied sequentially to dissociate the FFPE or OCT sample or specimen into single cells 1000 or nuclei. 1050. The physical and enzymatic/chemical dissociation systems can be separate from each other, or they can be co-located (e.g., acting upon the sample simultaneously or sequentially).

In some embodiments, the amount of dissociation is monitored at intervals during the dissociation or at the endpoint, and in some instances the viability is determined during processing using a Measurement Subsystem 500. The Measurement Subsystem 500 can be an optical imaging device to image cells or nuclei or tissue using brightfield, phase contrast, fluorescence, chemiluminescence, near-field, or other optical readouts, or an electrical measurement, such as an impedance measurement of the change in conductivity when a cell passes through a sensor, or other types of measurement.

The addition and movement of fluids can be performed by a Fluidic Subsystem 600. The Fluidic Subsystem 600 can use syringe pumps, piezopumps, on-cartridge pumps and valves, vacuum (negative pressure), pressure, pneumatics, or other components well known to one skilled in the art.

The Tissue Processing System 110 can be controlled by software in a Control Subsystem 700 which can be comprised of a user interface 740 through a monitor, embedded display, or a touch screen 730 to communicate with and control devices, modules, subsystems, instruments, and systems. In some instances the Control Subsystem 700 can include interfaces to smart devices, laboratory information management systems, other instruments, analysis software, display software, databases, email, and other applications. The Control Subsystem 700 can include control software 725 and scripts that control the operation and in some embodiments the scripts can be revised, created, or edited by the operator.

In another aspect provided herein is a device for the dissociation of a biological sample, the device comprising: (i) a biological sample or specimen 101; (ii) a cartridge 200 capable of dissociating tissue; (iii) an instrument to operate the cartridge 200 and provide fluids as needed (iv) a measurement module 500 such as an optical imaging to measure titer, clumping, and/or viability, (v) exchange of dissociation solution for buffer or growth media at the desired titer, and (vi) output vessels such as a chamber in the cartridge, 8 well strip tubes, microtiter plates, Eppendorf tubes, conical centrifuge tubes, or other vessels capable of receiving cell suspensions.

In another aspect provided herein is a device for the dissociation of a biological sample and the production of single-cell 1000 or nuclei 1050 suspensions or matched bulk nucleic acids 1010 or single cell libraries 1200 or matched bulk libraries 1210, the device comprising: (i) a chamber or area to input a biological sample or specimen either directly or in a device; (ii) a cartridge capable of dissociating tissue or specimen; (iii) an instrument to operate the cartridge and provide fluids as needed (iv) a measurement module such as an optical imaging to measure titer, clumping, and/or viability, necrosis, cytotoxicity, apoptosis, etc. (v) exchange of dissociation solution for buffer or growth media at the desired titer, (vi) the production of single-cell 1000 or nuclei 1050 suspensions or single cell libraries 1200, and matched bulk nucleic acid libraries 1210, in output vessels such as 8 well strip tubes, microtiter plates, Eppendorf tubes, a chamber in the cartridge, or other vessels capable of receiving cell suspensions.

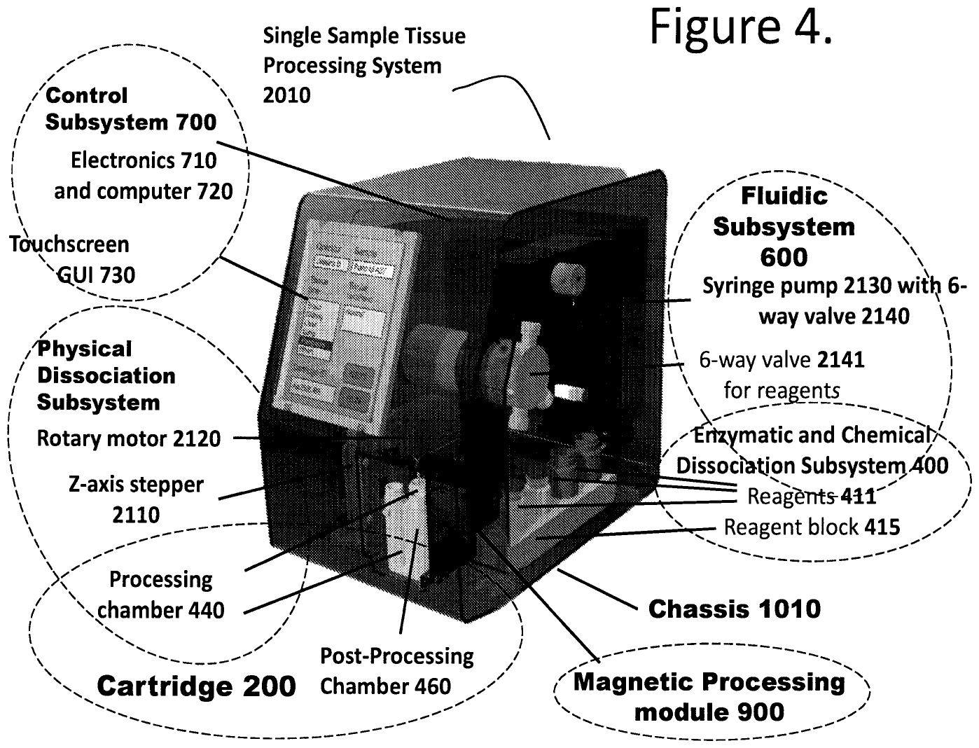

Referring to FIG. 4, a Magnetic processing module 900 can use magnetic processing of magnetic and paramagnetic particles or beads or surfaces or other sizes and shapes, referred to as beads, to separate single cells 1000, or cell types, or nuclei 1050, or other biocomponents comprised of subcellular components 1060, and biomolecules 1070 such as macromolecules 1071 and nucleic acids 1072, comprised of DNA 1073 and RNA 1074; proteins 1075; carbohydrates 1076; lipids 1077; biomolecules 1070 with multiple types of macromolecules 1071; metabolites 1078; and other biological components, including natural products 1079 for bioanalysis. In some embodiments the beads have a surface chemistry that facilitates the purification of the biologicals in conjunction with the chemical conditions. In other embodiments the beads have affinity molecules comprised of antibodies, aptamers, biomolecules, etc. that specifically purify certain biologicals such as cell types, nucleic acids, nuclei 1050, or other components of tissue or samples.

The basic elements of the Tissue Processing System 110 can be configured in multiple ways depending on the specimen(s) 101 or FFPE tissue specimens 150 or OCT tissue specimens 160 and analytes to be analyzed. In the following example, one of the numerous configurations are described in detail but in no way is the invention limited to these configurations as will be obvious to one skilled in the art. The Tissue Processing System 110 can accommodate many different types of specimens 101, comprised of fresh tissue; snap-frozen tissue; microtome slices (cryo, laser or vibrating) of tissue; fixed tissue; bulk material obtained by surgical excision, biopsies, fine needle aspirates; samples from surfaces, and other matrices, or FFPE tissue specimens 150.

The instant disclosure teaches how to produce a system that processes FFPE tissue specimens 150 and OCT tissue specimens 160 and other samples into preferentially nuclei 1050 or into single-cells 1000. The process may require adapting to the widely varying starting types of FFPE tissue specimens 150, with different requirements depending on the tissue, species, age, and state.

In the instant invention, many embodiments are possible and are incorporated by reference from patent application PCT/US2017/063811 filed Nov. 29, 2017 (Jovanovich, Chear, McIntosh, Pereira, and Zaugg, “Method and Apparatus for processing Tissue Samples”) and from patent application PCT/US19/35097 filed Jun. 1, 2019 (Jovanovich, Chear, Leisz, Eberhart, and Bashkin, “Method and Apparatus for processing Tissue Samples”); the contents of all are incorporated herein in their entirety as well as the number system used therein except where there is conflict the numbering herein predominates.

This disclosure describes how to automate, integrate, and importantly standardize the complete process to create single-nuclei 1050 in a single sample Tissue processing 110 system embodiment using a novel mechanism to retain the tissue and a novel cartridge design. It is obvious to one skilled in the art that multi-sample embodiments can be accomplished with the same instant invention. The Tissue Processing System 110 will greatly enable basic researchers, students, and translational researchers as well as clinicians and others with its ease of use and high performance.

### Single-Use Cartridge Designs

Cartridges 200 can be used to process tissue into single-cell 1000 suspensions or nuclei 1050 and are preferably single-use.

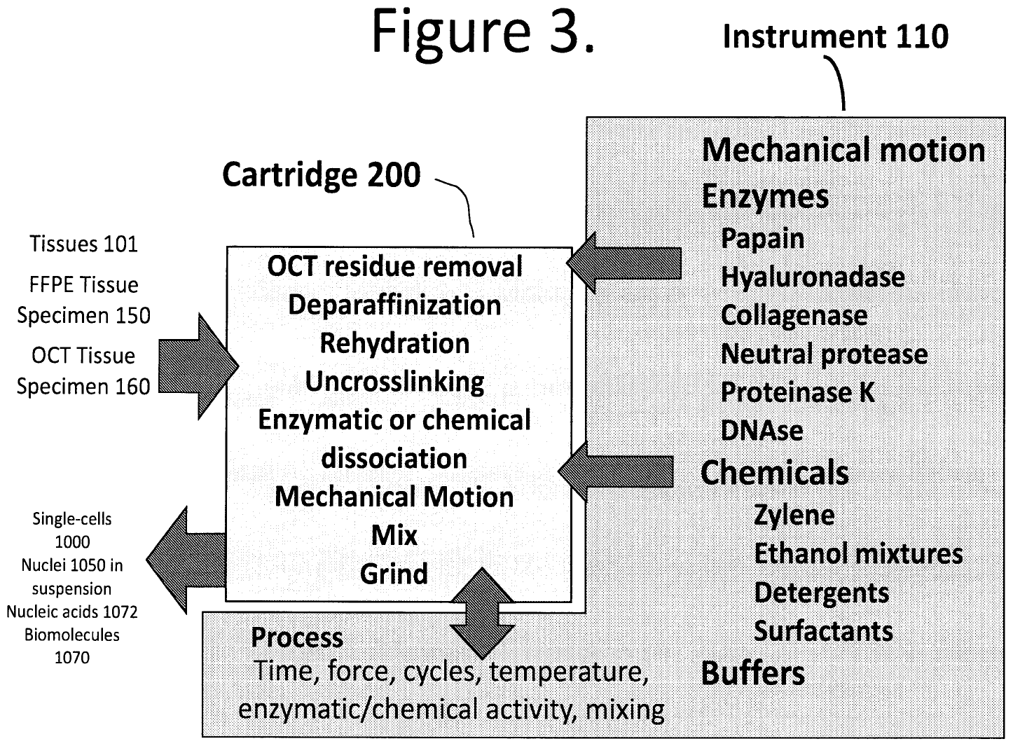

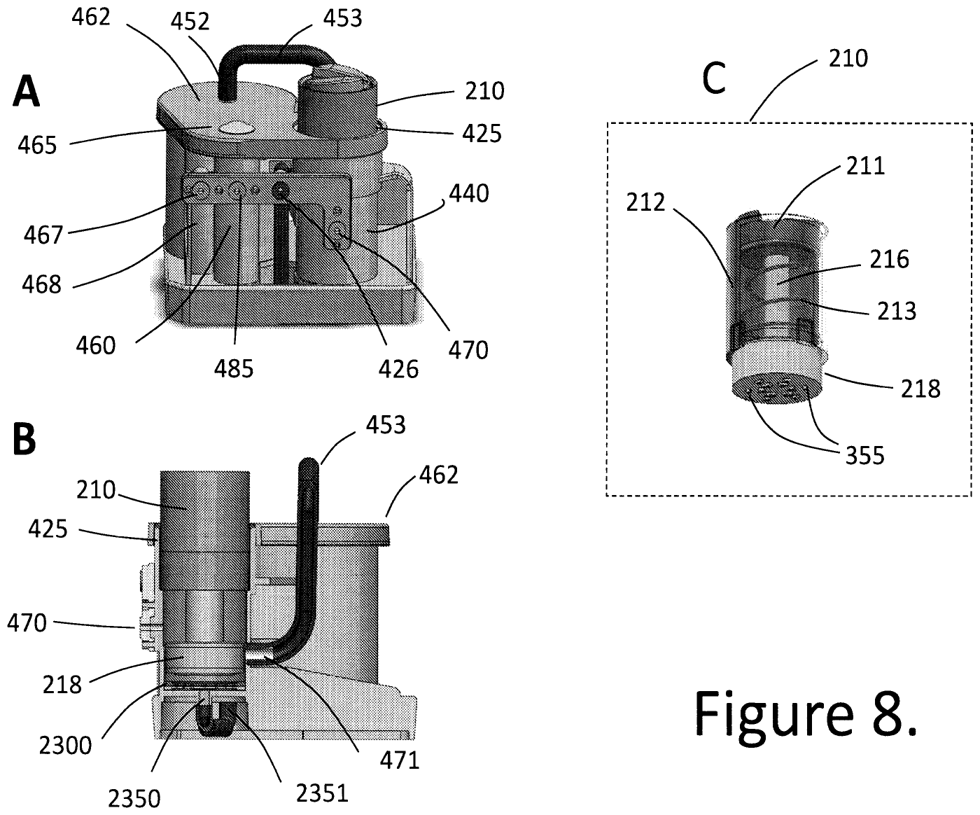

Referring to FIG. 3, cartridge 200 will input specimen 101 (e.g., fresh tissue) or FFPE tissue specimen 150 or OCT tissue specimen 160 and output singulated cells 1000 or nuclei 1050. The Tissue Processing System 110 as shown conceptually in FIG. 3 combines the mechanical disruption of specimen 101 on cartridge 200, adds reagents such as chemicals, detergents, enzymatic or chemical dissolution solutions 410 and other fluids according to the protocols, and controls sample movement, pressures, and temperature. The Tissue Processing System 110 can move or rotate mechanical tissue disruptor elements comprised of without limitation a syringe plunger, pestle, Dounce pestle, or grinder, using a z axis stepper 2110 with a rotary motor 2120 coupled through the cap 210. Referring to FIG. 8C, the term plunger is at times used to refer to combination of shaft/piston 216 and rotor 218 with optional disruption features (e.g., teeth) 355 with spring 213 in sheath 212.

In an embodiment, the mechanical tissue disruptor elements have features 355 on the bottom of the rotor or grinder that can mechanically disrupt tissue at the bottom or floor of processing Chamber 440 which in some embodiments may have complementary features 355 to aid in the disruption of the tissue. In some embodiments, the mechanical tissue disruptor elements does not have features 355 on the bottom of the rotor or grinder but can be flat and mechanically disrupt tissue against a flat surface at the bottom or floor of processing Chamber 440. Disruption also occurs in the ‘side gap’ between the rotor and the side wall of processing Chamber 440 in some embodiments.

It is desirable that disposable cartridge 200 process multiple types of preserved FFPE 150 or OCT 160 tissues with mechanical disruption and enzymatic or chemical dissociation that can be adjusted according to the tissue type and condition of the FFPE tissue, such as age, or chemical process. The cartridge 200 can be designed to process tissue as quickly and as gently as possible, not expose the operator to the tissue being processed, and be manufacturable at low cost. Multiple mechanical methods may be needed to accommodate the wide range of tissues and their individual requirements: designs are shown that can be readily adapted to multiple different mechanical disruption methods comprising variable orifice 490, grinding with rotating plungers 336, pestles 361, and straining and filtering using a plunger 362 as well as other mechanical methods without limitation.

Cartridges 200 can be designed for 3D printing, injection molding in plastics with single or double pulls and low labor assembly, or layered assembly of fluidic and other layers, combinations of methods, and other methods well known to one skilled in the art. Fluids can be delivered to cartridge 200 by pumps such as a syringe pump 2130 or by vacuum or can be preloaded onto cartridge 200 or many combinations. In some embodiments, flexible tubing 493 can connect chambers and creates simple pinch valves 491 to direct flow. In other embodiments, channels are created in the cartridge 200 and valves can be incorporated such as pneumatic valves, or other valves.

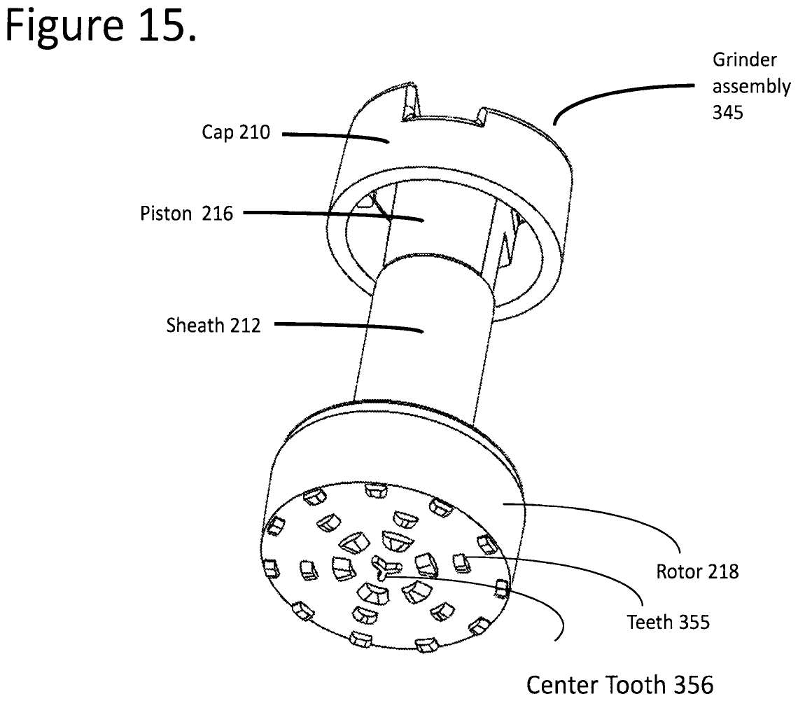

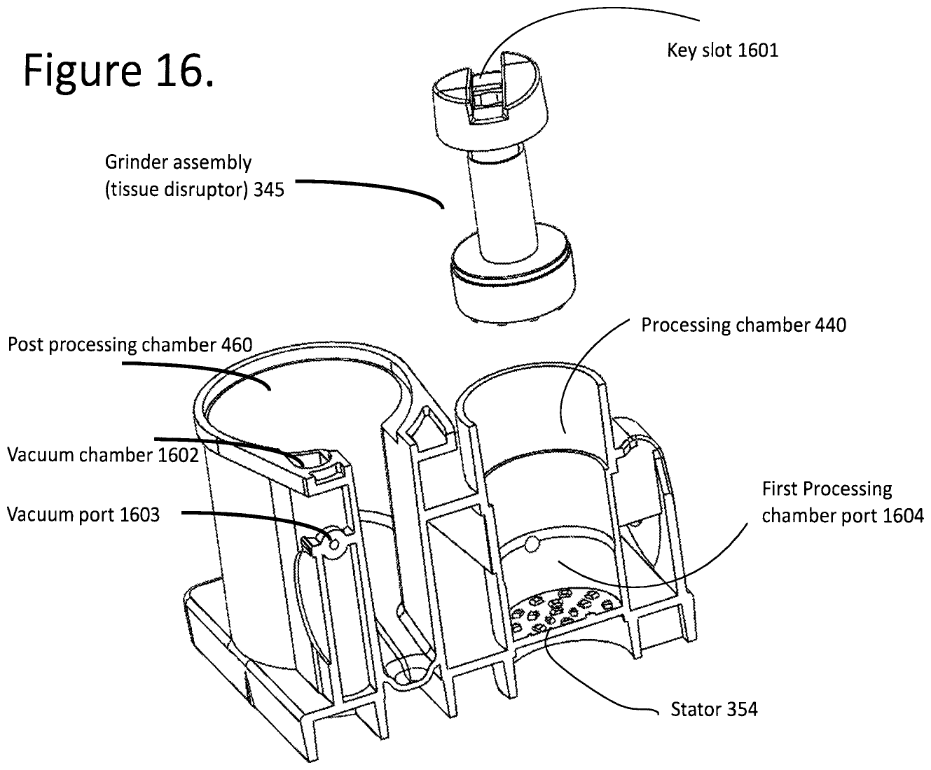

FIGS. 13-17 show an exemplary cartridge of this disclosure. FIG. 13 shows a cutaway view of an exemplary cartridge. The cartridge 200 includes a processing chamber 440 comprising a stator comprising teeth arranged in an annular array. The processing chamber further comprises a first processing port from which a cell, nuclei organelle suspension can be removed from the processing chamber. Also shown are post-processing chamber 460 and a vacuum chamber. The vacuum chamber 468 comprises a vacuum port 467. FIGS. 15 and 16 show an exemplary grinder assembly 345 of this disclosure. The grinder assembly 345 includes a plunger comprising a piston 216 and a rotor 218 positioned at an end of the piston. The rotor comprises on a bottom surface, grinding elements, e.g., teeth 355, including a central tooth 356 and an annular array of three rings of teeth. The teeth 355 can have a blunt or sharp shape. They may take the shape of a trapazoid in cross section. The outermost ring of teeth 355 is positioned at the edge of the rotor. The grinding assembly 345 further includes a sleeve or sheath 212 around the piston 216. The grinding assembly 345 further includes a cap 210 to position the plunger in the processing chamber. The cap 210 further comprises a slot or other mechanism configured to engage a key of an actuator to actuate the grinder. Not shown here is a spring which biases the rotor 218 toward the cap 210 so that positive pressure must be asserted on the plunger by the actuator to press the rotor 218 against the stator. The annular rings of teeth 355 in the rotor and the stator are positioned complementary to one another so that when the grinder is pressed against the stator the rings of the stator mesh with the rings of the rotor (e.g., are staggered against). That is, in an exemplary embodiment, teeth in the stator do not touch teeth in the rotor. This configuration facilitates rotation of the rotor against the stator so that teeth from one part do not collide with teeth from another part. The number of rings of teeth in each of the rotor and the stator can be determined by a skilled artisan. Factors influencing the determination include the total surface area of the stator and the face of the rotor, as well as the size of the teeth. In certain embodiments the number of rings of teeth in the stator and/or the rotor 218 can be any of none, one, two, three, four, five, or six. In one embodiment teeth can have a trapezoidal cross-section. The processing chamber can have a cylindrical shape. The stator can have a radius between, for example, 5 mm and 25 mm, e.g., about 12 mm. The processing chamber can have a volume less than 1 ml, or between about 1 mL and 50 mL, for example, between about 10 mL and about 30 mL, e.g., about 15 mL. The rotor and the sidewalls of the processing chamber can be configured so that when the plunger is inserted into the processing chamber there is a gap between the sidewall of the processing chamber and an edge of the rotor. The size of the gap can be optimized to allow passage of whole cells, nuclei or organelles between the sidewall and the rotor. In certain embodiments, the teeth can have a height of about 500 microns and a width of about 1 mm to 2 mm.

Spin rates for the dissociation can be 10-200 rpm. Total revolutions of the grinding element can be 5-500. In an exemplary protocol, the spin rate is about 45 rpm (slow) or about 150 rm (fast), with about 4 seconds of revolution, about 1-2 second pause, then about another 4 seconds, then repeat (about 16 seconds total rotation time) at each vertical displacement step of the stepper motor, sequentially going lower towards the bottom of the cartridge, about 9 vertical displacements in all, and at the bottom-most step, there are about 3 repetitions of the rotation periods rather than 2.

### Tissue Processing System Embodiment

In one embodiment of the Sample processing System 50 as a Tissue Processing System 80, as shown in FIG. 1, the Tissue Processing System 110 can perform powerful integrated tissue-to-genomics or sample-to-other answer (genomic, proteomic, metabolomic, or epigenetic, multi-omics, etc.) analysis functionality for scientists to simply and standardize the production and or analysis of single-cell 1000 or nuclei 1050 suspensions, affinity purified single cells 1100, affinity purified nuclei 1105, nucleic acids 1072, and bulk libraries 1210 from solid or liquid tissues. As will be obvious to one skilled in the art, the biological materials produced such as single cells 1000, nuclei 1050, nucleic acids 1072, single cell libraries 1200, single nuclei libraries 1250, bulk libraries 1210, or other biocomponents comprised of subcellular components 1060, or biomolecules 1070 such as macromolecules 1071 and nucleic acids 1072, comprised of DNA 1073 and RNA 1074, can also be used for many genomic, cell biology, proteomics, metabolomics, and other analytical methods.

The Tissue Processing System 110 can integrate the preparation of biological materials from FFPE tissue specimens 150 or OCT tissue specimen 160 with measurement subsystems 500 that perform an analysis selected from one or more of: DNA or RNA sequencing, next generation DNA or RNA sequencing, next next generation DNA or RNA sequencing of nucleic acids and their adducts such as epigenetic modifications; nanopore sequencing of nucleic acids and their adducts; single cell DNA sequencing of nucleic acids and their adducts; single nuclei RNA sequencing of nucleic acids and their adducts; PCR, digital droplet PCR, qPCR, RT-qPCR; genomic analysis, gene expression analysis, gene mapping, DNA fragment mapping; imaging including optical and mass spectrometry imaging; DNA or RNA microarray analysis; fluorescent, Raman, optical, mass spectrometery and other detection modalities of nucleic acids acids and their adducts with and without labels; proteomic analysis including fluorescent, Raman, optical, mass spectrometery, protein sequencing, and other detection modalities of proteins and peptides and their adducts and modifications with and without labels; carbohydrate characterization and profiling including sequencing, fluorescent, Raman, optical, mass spectrometery, and other detection modalities of carbohydrates and their adducts and other covalent polymers with and without labels; lipid characterization and profiling including sequencing, fluorescent, Raman, optical, mass spectrometery, and other detection modalities of lipids and their adducts and other covalent polymers with and without labels; flow cytometry; characterization of cells and profiling including fluorescent, Raman, optical, mass cytometery, and other detection modalities of cells and their adducts and other covalent polymers with and without labels; metabolic profiling including sequencing, fluorescent, Raman, optical, mass spectrometery, and other detection modalities of metabolites and their adducts and other covalent polymers with and without labels; functional analysis including protein-protein interactions, protein-lipid interactions, protein-DNA interactions, RNA-DNA interactions, and other interactions between molecules derived from biological materials, with and without labels;

bioinformatic analysis of cells, organelles, and biomolecules; and mass spectrometry and other analytical methods. In some embodiments the measurement system 500 can be physically integrated and fluids transferred by robotic pipetting, fluid flow through tubing or capillaries, centrifugal methods, or other methods.

Referring to FIG. 4, in this preferred embodiment mechanical and enzymatic dissociation is performed in single-use cartridges 200 in one or more processing chambers 440 to produce nuclei suspensions 1200, single-cell suspension 1000 or, nucleic acids 1072, biomolecules 1070, subcellular components 1060, or other products. The samples can then be processed in the one or more post-processing chamber(s) 460 by optional bead-based affinity purification of cell types by surface antigens to produce affinity purified single-cell suspensions 1100 or nuclear suspensions by nuclear antigens 1105 or nucleic acids 1072, biomolecules 1070, subcellular components 1060 can be further processed into purified mRNA, NGS libraries, or other sample types. In some embodiments, one or more of the processing 440 and post-processing chambers 460 and strain chambers 450 and vacuum trap chambers 468 and waste chambers 430 or other chambers can be combined.

### Computer Systems

Models provided herein can be executed by programmable digital computer.

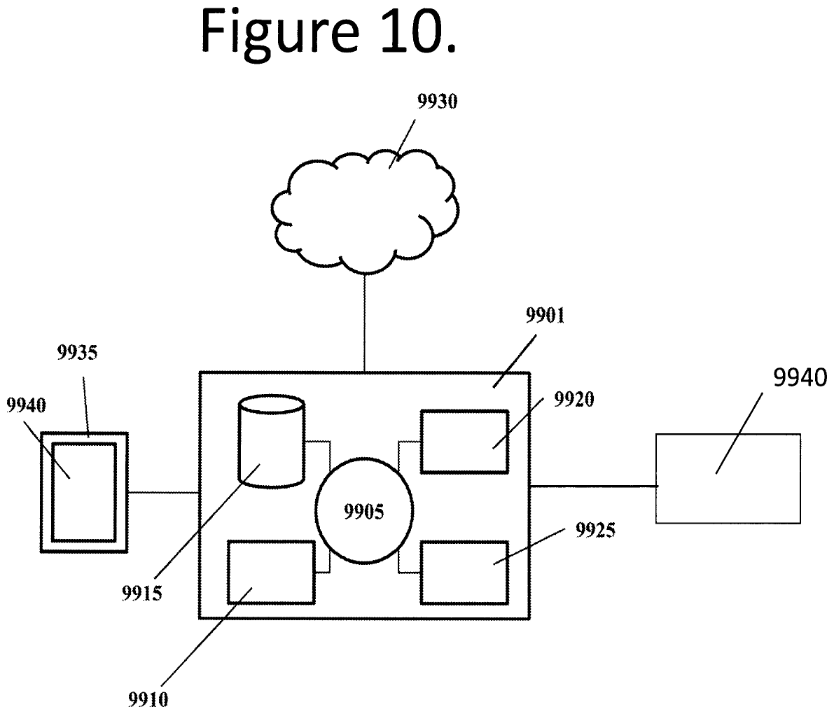

FIG. 10 shows an exemplary computer system. The computer system 9901 includes a central processing unit (CPU, also “processor” and “computer processor” herein) 9905, which can be a single core or multi core processor, or a plurality of processors for parallel processing. The computer system 9901 also includes memory or memory location 9910 (e.g., random-access memory, read-only memory, flash memory), electronic storage unit 9915 (e.g., hard disk), communication interface 9920 (e.g., network adapter) for communicating with one or more other systems, and peripheral devices 9925, such as cache, other memory, data storage and/or electronic display adapters. The computer readable memory 9910, storage unit 9915, interface 9920 and peripheral devices 9925 are in communication with the CPU 9905 through a communication bus (solid lines), such as a motherboard. The storage unit 9915 can be a data storage unit (or data repository) for storing data. The computer system 9901 can be operatively coupled to a computer network (“network”) 9930 with the aid of the communication interface 9920. The network 9930 can be the Internet, an internet and/or extranet, or an intranet and/or extranet that is in communication with the Internet. The network 9930 in some cases is a telecommunication and/or data network. The network 9930 can include one or more computer servers, which can enable distributed computing, such as cloud computing.

The CPU 9905 can execute a sequence of machine-readable instructions, which can be embodied in a program or software (code). The instructions may be stored in a memory location, such as the computer readable memory 9910. The instructions can be directed to the CPU 9905, which can subsequently program or otherwise configure the CPU 9905 to implement methods of the present disclosure.

The storage unit 9915 can store files, such as drivers, libraries, and saved programs. The storage unit 9915 can store user data, e.g., user preferences, log files, video or other images, and user programs. The computer system 9901 in some cases can include one or more additional data storage units that are external to the computer system 9901, such as located on a remote server that is in communication with the computer system 9901 through an intranet or the Internet.

The computer system 9901 can communicate with one or more remote computer systems through the network 9930.

Methods as described herein can be implemented by way of machine (e.g., computer processor) executable code stored on an electronic storage location of the computer system 9901, such as, for example, on the computer readable memory 9910 or electronic storage unit 9915. The machine executable or machine-readable code can be provided in the form of software. During use, the code can be executed by the processor 9905. In some cases, the code can be retrieved from the storage unit 9915 and stored on the memory 9910 for ready access by the processor 9905. In some situations, the electronic storage unit 9915 can be precluded, and machine-executable instructions are stored on memory 9910. The code can be used to communicate and issue instructions to electronic devices, e.g., circuit boards 9940, modules, or subsystems, on the instrument, for example, the rotary DC motor relay board 2134 or the heater relay board 2240 driving peltier 1420 to accomplish tasks such as rotating a motor or controlling the temperature of the cartridge 200.

The computer system 9901 can communicate with one or more remote computer systems through the network 9930.

Methods as described herein can be implemented by way of machine (e.g., computer processor) executable code stored on an electronic storage location of the computer system 9901, such as, for example, on the computer readable memory 9910 or electronic storage unit 9915. The machine executable or machine-readable code can be provided in the form of software. During use, the code can be executed by the processor 9905. In some cases, the code can be retrieved from the storage unit 9915 and stored on the memory 9910 for ready access by the processor 9905. In some situations, the electronic storage unit 9915 can be precluded, and machine-executable instructions are stored on memory 9910. The code can be used to communicate and issue instructions to electronic devices, e.g., circuit boards 9940, modules, or subsystems, on the instrument, for example, the rotary DC motor relay board 2134 or the heater relay board 2240 driving peltier 1420 to accomplish tasks such as rotating a motor or controlling the temperature of the cartridge 200.

Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks.

The computer system 9901 can include or be in communication with an electronic display 9935 that comprises a user interface (UI) 9940 for providing, for example, input parameters for methods described herein. Examples of UIs include, without limitation, a graphical user interface (GUI) and web-based user interface.

### Example: A Single-Sample Tissue Processing System to Single Cell and Nuclei Suspensions

The Tissue Processing System 110 can mechanically disrupt tissue and enzymatically dissociate and reverse crosslinks of the disrupted tissue in a cartridge 200 into single cells or nuclei 1050. As shown in FIG. 4, a Single Sample Tissue Processing System 2010 can combine the Physical Dissociation Subsystem 300 and the Enyzmatic and Chemical Dissociation Subsystem 400 to produce single-cell 1000 or nuclei 1050 suspensions. The instrument provides the mechanical motion and fluidics to the cartridge which in turn mechanically and enzymatically or chemically process the FFPE tissue specimen 150 into single cells 1000 or nuclei 1050. Multiple reagents 411 can be stored on the instrument or reagent module 1430 with cooling as needed.

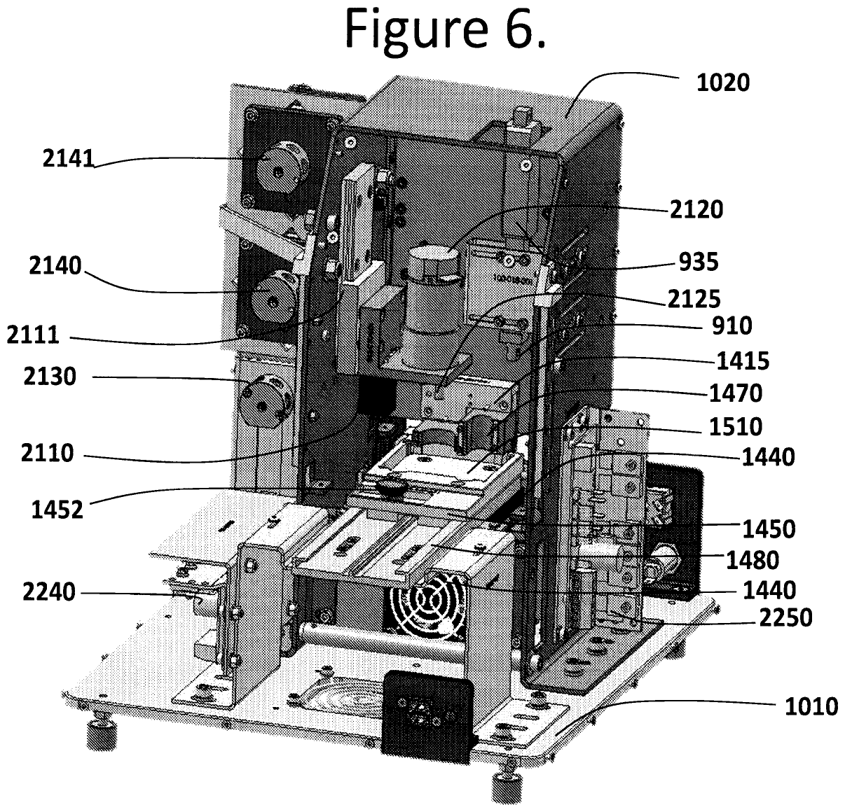

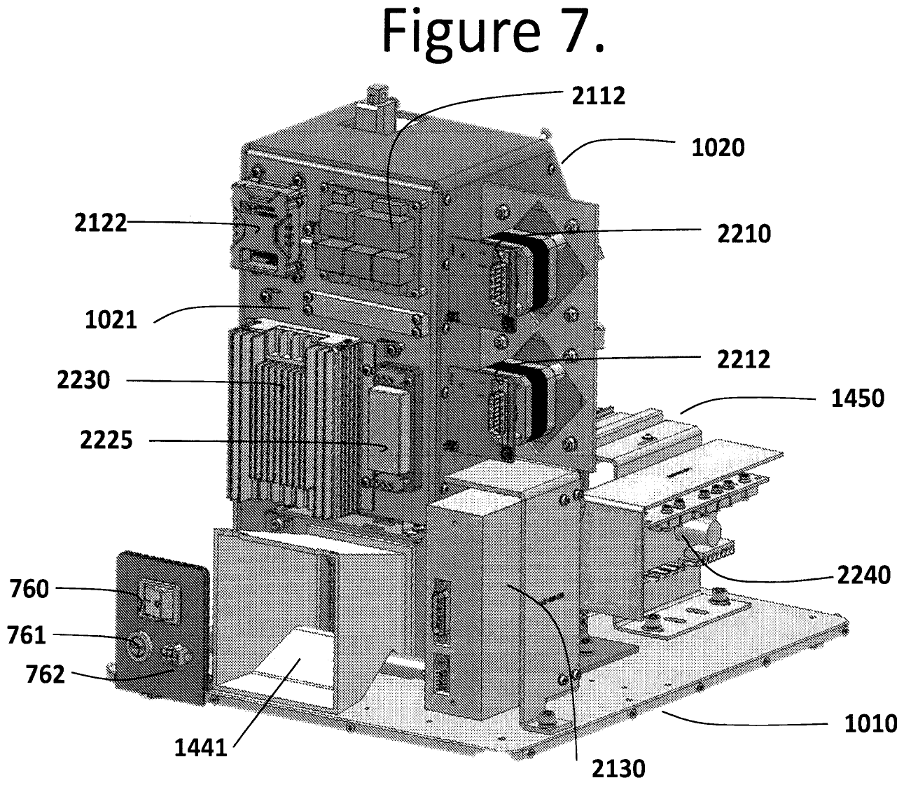

A 3D CAD representation of one embodiment of a Single-Sample Tissue processing 2010 design packaged with a ‘skin’ is shown in FIG. 4 and another embodiment is shown in FIGS. 6 and 7. Both embodiments have a two axis mechanical motion (Z axis stepper 2110 and rotary motor 2120) integrated with fluidics based on a syringe pump, for example, with 1.6 μL resolution with a six-way valve (C2400MP, TriContinent) controlled by control software 725.

Referring to FIG. 4, a computer 720 with an operating system, for example, such as Windows 10 and 85 Gbytes HD (Beelink, AP42) can run control software 725 to control the system with display on a 10″ touchscreen 730 (eleduino, Raspberry Pi10) or on a tablet 750 such as a Windows Surface Pro 6. Chassis 1010 provides the framework to mount components and the exterior case of the system.

The embodiment of the Single-Sample Tissue Processing System 2000 shown in FIG. 4 has a fluidic subsystem 600 with a single syringe pump 2130 with a single six-way valve 2140 to supply liquids, pressure, or vacuum to cartridge 200 from reagent block 415. In one embodiment, cartridge 200 has two processing chambers 440 and a single post-processing chamber 460. In an embodiment, magnetic processing module 900 can apply magnetic force to cartridge 200 under software control to enable the use of paramagnetic beads, paramagnetic surfaces, paramagnetic nanoparticles, and other magnetic or paramagnetic particles to purify and analyze single cells 1000, nuclei 1050, nucleic acids 1072, biomolecules 1070, subcellular components 1060, or other products.

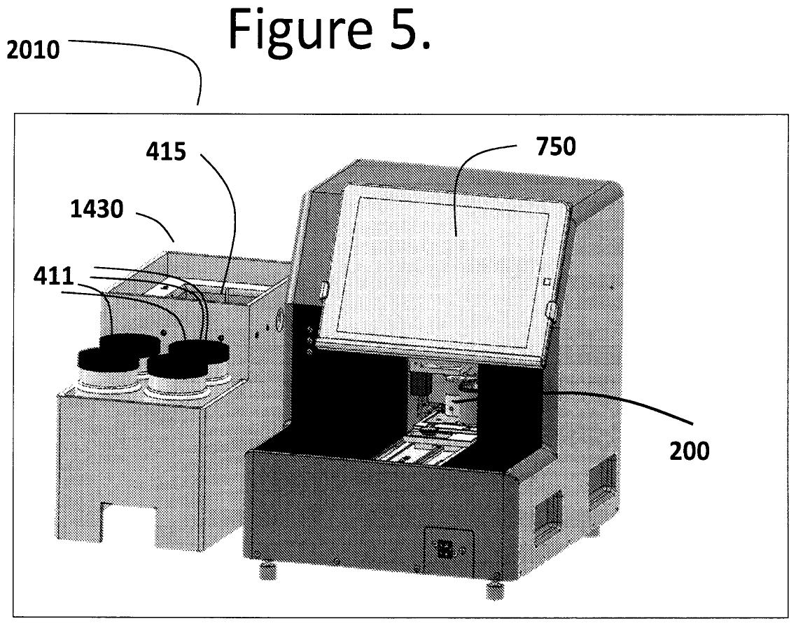

An embodiment of the Single-Sample Tissue processing 2010 with a case on is shown in FIG. 5. This embodiment has a reagent module 1430 which can be separate from Single Sample Tissue processing Instrument 2010 as shown in FIG. 4 with power and control provided by Single Sample Tissue processing Instrument 2010 or a separate power source and processor can be used, or as shown in FIG. 4 reagent module 1430 be integrated inside a single instrument case.

Referring to FIG. 6, in an embodiment, Single Sample Tissue processing Instrument 2010 has z-axis stepper motor 2110, which may have an optional encoder, that controls the vertical position of rotary motor 2120 mounted on z-axis stepper slide 2111 attached to the inverted ‘U’ shaped structural frame 1020 mounted on chassis 1010. A force gauge can be incorporated into the z-stage stepper 2110 to provide force-feedback control of the mechanical force on the specimen 101 or below cantilevered cartridge slide 1450; this can help ensure very gentle mechanical processing steps and prevent application of high force by the rotor 218 onto the bottom of processing chamber 440. Syringe pump 2130 connects fluidically with tubing or capillaries or microchips or other fluidic connectors with six-way valve 2141 and six-way valve 2142 to supply reagents, pressure, or vacuum to cartridge 200 (not shown) from reagent module 1430.

Cartridge 200 is placed into cartridge receiver tray 1510 on cartridge slide 1450 which is designed to hold cartridge 200 precisely, with the center of processing chamber 440 concentric with the center of rotary motor shaft 2121 of rotary motor 2120 within a distance or 1 or, 5, or 10, or 15, or 20, or 25, or 50, or 100, or 250 μm, or more when inserted by moving cartridge 200 in cartridge receiver tray 1510 on cartridge slide 1450 on cartridge slide rail 1480 until spring-loaded cartridge slide knob 1452 locks into place into a hole in cartridge slide 1450 with cartridge 200 held in place near or in contact with the thermal transfer plate 1470 and making fluidic connections with the pogo pins 1415 of cartridge interface 1500.

The temperature regulating subsystem 1475 can set the thermal transfer plate 1470 to a given temperature by cartridge Peltier 1440 or other temperature regulating device such as strip resistive heaters, circulating fluids, etc. to set the cartridge temperature in the processing chamber 440 and post-processing chamber 460 under control of board 2250. In some embodiments, the temperature of processing chamber 440 and post-processing chamber 460 can be set independently. In some embodiments the temperature regulating system can use a thermocouple, or thermister, or IR camera to set the temperature of the thermal transfer plate 1470 or the outside of cartridge 200.

In an embodiment, fluidic ports on cartridge 200 dock with spring-loaded pogo pins 1415 to connect fluids, gases, or vacuum to cartridge 200 on cartridge insertion. In another embodiment, pogo pins 1415 or canula 1416 are moved to connect with cartridge 200 after cartridge insertion. In another embodiment, canula 1416 connected to fluidic lines from syringe pump 2130 are held rigidly attached to the thermal transfer plate 1470 or other part of instrument and cartridge 200 has flexible materials on cartridge ports that seal with the canula(s) 1416 after cartridge insertion, as described below. Cartridge ports are ports opening out of a cartridge. A cartridge port may communicate directly with a chamber by being a port in the chamber, or indirectly, e.g., through another chamber comprising the port and communicating with the chamber in question.

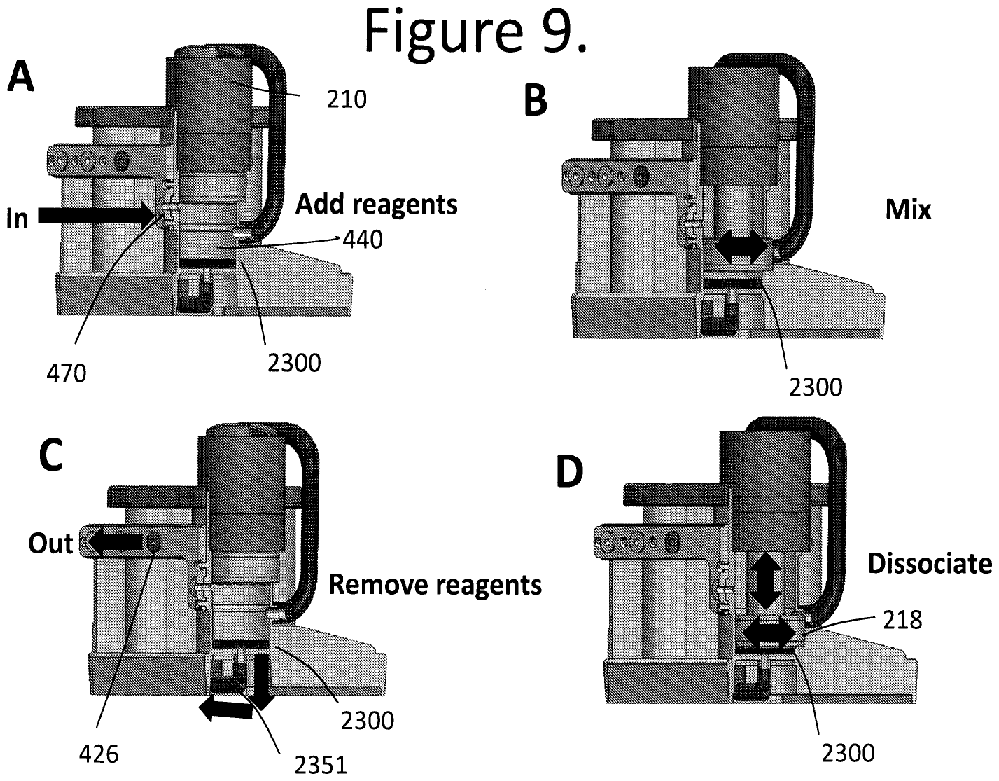

The embodiment of the single-sample Tissue Processing System 2010 shown in FIG. 6 has a Magnetic processing Module 900 and magnet 910 is moved by magnetic actuator 935 mounted on inverted ‘U’ shaped structural frame 1020 under control of control software 725 using controller 2122. Magnet 910 can be far from cartridge 200 as shown in FIG. 9 and not interact with any magnetic beads 685 in cartridge 200 or in an extended position magnet 910 is moved to be near cartridge 200 for magnetic capture and processing of magnetic beads 685. Many embodiments of configurations of the geometric relationship of the Magnetic processing Module 900 and magnet 910 and cartridge 200 are possible.

Referring to FIG. 7, in an embodiment, the Single-Sample Singulator System 2000 has a back structural frame 1021 on structural frame 1020 that mounts electronics 710 comprising rotary motor controller 2122, z-axis stepper controller 2112, 24 V to 5 V step down power supply 2230 and 24 V to 12 V step down power supply 2225. Power can be supplied to single-sample Tissue processing 2010 by plugging a 24 V power supply into plug 762 connecting to fuse 761 and power switch 760. Six way valves 2141 and 2142 are controlled by boards 2210 and 2212. Reagent Peltier relay board 2240 can control reagent Peltier 1420.

Systems that process one or more cartridges simultaneously are within the scope of the present invention. The cartridge 200 can have one or more processing Chamber(s) 440 and none, one, or more Post-Processing Chamber(s) 460 as well as none, one or more other chambers such as cartridge waste chamber 435 or vacuum trap chamber 468.

In one embodiment, illustrated in FIG. 8, cap 210, alternatively referred to as a tissue disruptor, is placed on top of processing chamber 440 after specimen 101 is added into processing chamber 440 of cartridge 200. After cartridge 200 is inserted into the instrument, pogo pins 1415, canula 1416, or other fluidic connectors can connect with none, one, or more of cartridge ports 470 to supply reagents to processing chamber 440, cartridge port 485 to supply reagents or vacuum to post-processing chamber 460, cartridge vacuum trap port 467 to supply vacuum to vacuum trap chamber 468, or cartridge waste port 2355 to supply vacuum or reagents to cartridge waste line 2351.

An embodiment of cartridge 200 for processing tissue specimen 150 illustrated in FIG. 8 fluidically connects processing chamber 440 to post-processing chamber 460 using fluidic line 453, which can be tubing, connecting from processing chamber nipple 471 to lid nipple 452 positioned over strainer 2711 inserted into post-processing chamber 460. In other embodiments, no strainer can be used or strainer 2711 can be incorporated as an in-line filter, for example in a swinney filter holder 347 attached to the output of processing chamber 440 or in fluidic line 453 or attached to lid 462. In an embodiment, dual or triple or more filters are used in strainer 2711, for example, a 145 micron filter followed by a 40 micron filter followed by a 20 micron filter; other combinations are envisioned.

Lid 462 produces a vacuum tight seal of post-processing chamber 460 and vacuum trap chamber 468 when cap 465 is sealed on lid 462. Lid 462 can be attached to cartridge body 201 by ultrasonic welding, glue, epoxy, adhesives, and other methods to produce a vacuum tight seal. The permanent attachment of lid 462 ensures single usage of cartridge 200 to eliminate cross sample contamination by preventing changing of strainer 2711.

In some embodiments, cartridge 200 can have on-cartridge valves which can be pinch valves 491 on fluidic lines such as fluidic line 453 which the instrument actuates to open and close lines, or by using a ‘T’ junction and two lines, rout fluids down different paths such as to on cartridge waste or to an a optics imaging system 520, or to multi'omics processing of another workflow or analysis method. In another embodiment, fluidic lines such as fluidic line 453 can be partially closed to create a variable orifice 2160 that can disrupt partially dissociated tissue. Actuators can open and pinch close tubing in the cartridge 200, or operate the variable orifice 2160 using variable orifice device 2150 when desired. In other embodiments, cartridge 200 can have on-cartridge valves which can be miniaturized pneumatic valves, or microvalves. In some embodiments, microfluidics or microchips are used for fluidic lines. In an embodiment there are no valves on the cartridge 200 with all fluidic control from the instrument.

Referring to FIG. 8A, when vacuum is applied to vacuum trap port 467 or to reagent port 485, liquids including single cell suspensions 1000, nuclei 1050, and other subcellular components 1060, and biomolecules 1070 are pulled from processing chamber 440 through fluidic line 453 and strainer 2711 into strain drain 451 and into output collector region 461 of post-processing chamber 460. Strainer 2711 can have pore sizes such as 2, 5, 10, 15, 20, 25, 30, 40, 50, 70, 100, 125, 200 μm, or larger to filter the suspension of biological material. Muiltiple in-line or stacked strainers 2711 can be employed to successively remove different sized components of the dissociated tissue specimen 110. Cap 210 with cap coupler 211, and head 218 (also referred to as “rotor” 253) is shown ready to be inserted into sample inlet port 425. Head 218 can have a surface for disrupting tissue that can comprise raised features 355 that aid in mechanically disrupting a tissue, organ, microtissue 6001, organoid 6002 or other biological material.

Referring to FIG. 8B and FIG. 8C, the cap coupler (also referred to as “drive head”) 211 is held inside cap sheath 212 which in one embodiment has cap sheath hole 214. Cap coupler 211 is attached to cap shaft 216 which passes through cap sheath hole 214 and is attached to the head 218 which can be a rotor 353 with grinding teeth 355. The assembly of cap coupler 211 attached to cap shaft 216 and head 218 are referred to as a plunger 336 which is a type of moveable mechanical tissue disruptor 345.

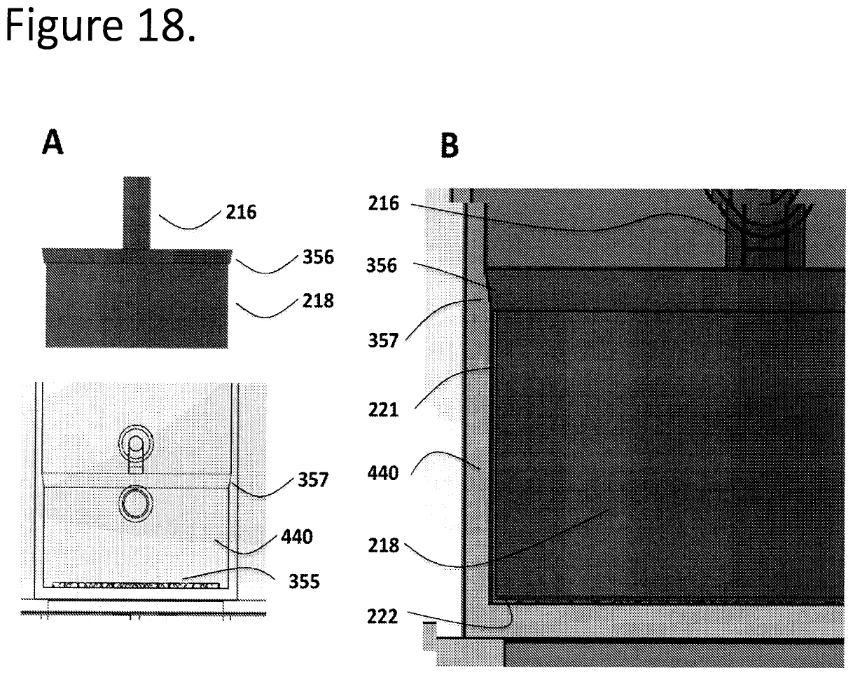

Referring to FIG. 18A, in a preferred embodiment, head 218 attached to cap shaft 216 has a outwardly annular beveled head feature 356 designed to improve centricity of head 218 inside processing chamber 440 and thereby the uniformity of side gap 221 at the bottom of travel. When z-axis stepper motor 2110 lowers and cap coupler 211 is pushed down by rotary motor coupler 2125, head 218 will lower until outwardly annular beveled feature 356 engages with inwardly annular beveled preprocessor chamber feature 357 on the inside wall of processing chamber 440 to produce a centered head 218 as shown in FIG. 18B. The centering of head 218 will produce a uniform side gap 221 between head 218 and the inner wall of processing chamber 440. In addition, if the height of head 211 is less than the height of the processing chamber 440 below inwardly beveled feature 357, the engagement of outwardly annular beveled head feature 356 with inwardly annular beveled preprocessor chamber feature 357 will set a uniform bottom gap 222. The size of the side gap and the bottom gap can be optimized for different cell types or for different sized nuclei or subcellular organelles, or multicellular structures such as intestinal crypts. In addition, to allow passage of disrupted tissue when head 218 is seated on inwardly annular beveled preprocessor chamber feature 357, the inwardly annular beveled preprocessor chamber feature 357 can be fluted to have sections with the same or different depths. The side gap 221 between the head 218 of moveable mechanical disruptor 345 and the inside wall is preferably greater than or equal to 1 μm, or 2 μm, or 5 μm, or 10 μm, or 15 μm, or 20 μm, or 25 μm, or 30 μm, or 40 μm, or 50 μm, or 75 μm, or 100 μm, or 150 μm, or 200 μm, or 250 μm, or 500 μm, and 1000 μm or more, as well as any size in between. In a preferred embodiment, the side gap 221 is greater than 50 microns and less than 150 microns for nuclei and other subcellular organelles and greater than 75 microns less than 250 microns for cells. A gap size for isolation of nuclei can be, for example, between about 30 microns and about 200 microns, e.g., about 40 microns and about 150 microns, or about 100 μm to about 125 μm. A gap size for isolation of cells can be, for example, between about 50 μm in about 400 μm, e.g., about 200 μm to about 300 μm, or about 250 μm. The bottom gap 222 between the bottom of head 218 of moveable mechanical disruptor 345 and the bottom of processing chamber 440 is preferably greater than or equal to 1 μm, or 2 μm, or 5 μm, or 10 μm, or 15 μm, or 20 μm, or 25 μm, or 30 μm, or 40 μm, or 50 μm, or 75 μm, or 100 μm, or 150 μm, or 200 μm, or 250 μm, or 500 μm, and 1000 μm or more, as well as any size in between. In some embodiments, different heads can be selected to be used with the same diameter processing chamber 440 to produce different side gaps 221 or bottom gaps 222 to simplify manufacturing and inventory management requirements. A bottom gap between a flat surface of the head and the flat bottom surface of the processing chamber can also be limited by the position of the flutes, or half domes, or other structures that prevent or define gaps between a flat surface of the head and the flat bottom surface of the processing chamber.

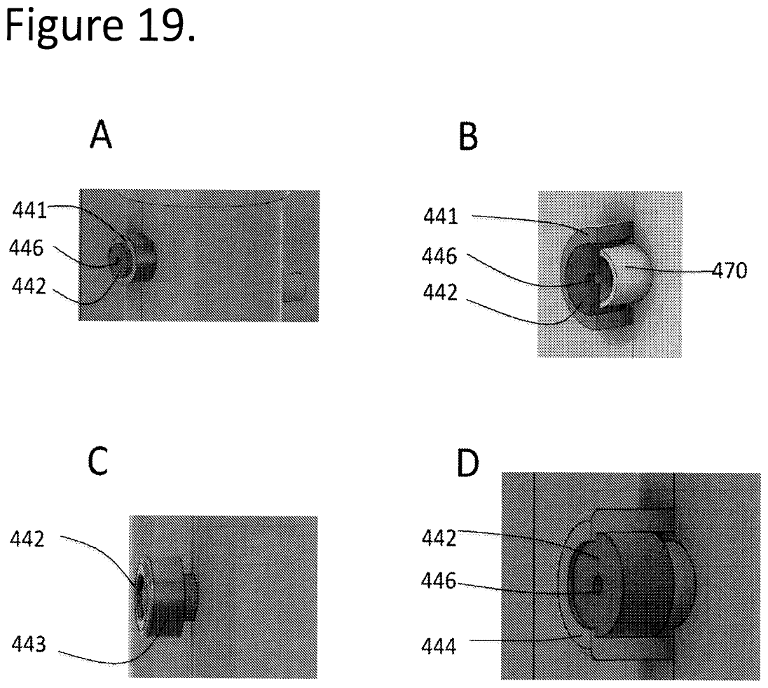

Referring to FIG. 19, none, one, or more of the ports to cartridge 200 can have flexible or low durometer port covers 442, for example without limitation 40 to 100 durometer. As illustrated in FIG. 15A and in cutout FIG. 15B, port cover 442 can be inserted into the space between the port and port cover retaining cylinder 441 to secure the port cover 442 in place over, for example as shown, reagent addition port 470. A fluidic canula 1416 or fluidic pogo pin 1415 with an outside diameter larger than port cover center hole 446 can engage the port covered by port cover 442 and, because of the relatively low durometer, the port cover 442 will be deformed by fluidic canula 1416 or fluidic pogo pin 1415 to create a seal around the fluidic canula 1416 or fluidic pogo pin 1415. In some configurations, the deformation can be used to eliminate the need for springs and the use of the fluidic pogo pin 1415 can be replaced by a non-movable fluidic canula 1416. FIG. 15C shows port cover 442 retained by crimp seal 443. FIG. 19D shows port cover 442 retained by forming port cover retaining cylinder 442 higher than the port cover 442 and melting the port cover retaining cylinder 442 to form a heat stake lip 444 that retains port cover 442.

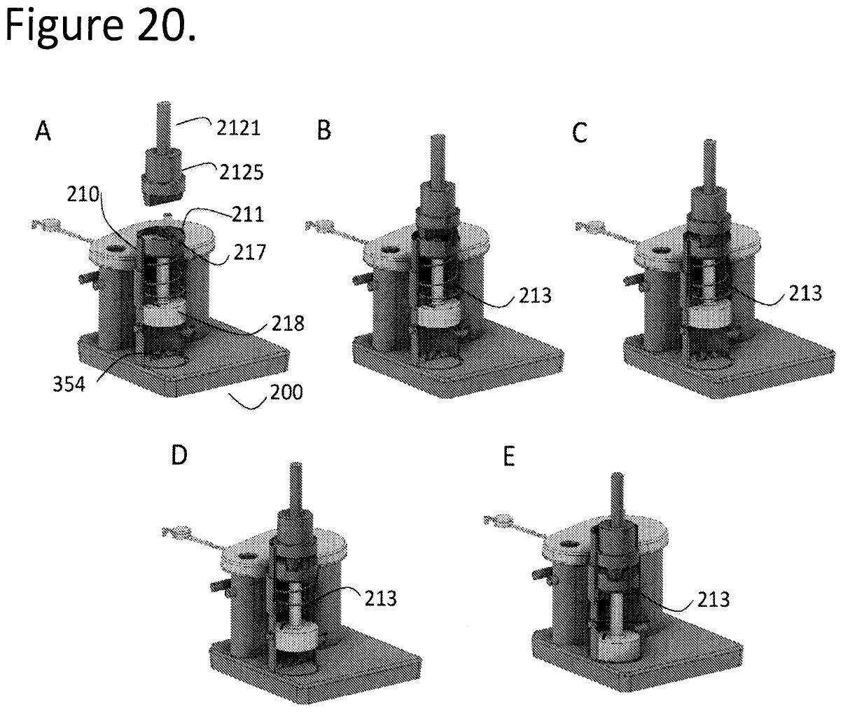

In a preferred embodiment the Single Sample Singulator Instrument 2050 has an actuator for mechanical processing that has a stepper motor 2110 that controls the vertical position of rotary motor 2120 and rotary motor shaft 2121 attached to rotary motor coupler 2125 that in turn can mechanically couples with cap coupler 211 of the cap 210 when inserted into cartridge 200. The coupler can have a drive head that takes any appropriate form, such as a slot, a phillips head, a quadrex, atri-wing, aspanner or a hex. Rotary motor coupler 2125 has one or more facets that reversibly engage cap coupler 211 by actions such as moving downward and slowly rotating. As shown in FIG. 20A, in a preferred embodiment, rotary motor coupler 2125 has a single blade to engage cap coupler 211 in stepper motor 2110 lowers, the rotary motor coupler 2125 attached to rotary motor shaft 2121 engages cap coupler 211 in cap 210 and if the rotary motor coupler 2125 is not lined up with cap coupler groove 217, the rotary motor coupler 2125 can not directly insert into the cap coupler groove 217. In a preferred embodiment, cap coupler 211 has two surfaces on either side of cap coupler groove 217 which slope in opposite directions across the cap coupler 211 such that each side has a higher and lower wall on either side of cap coupler groove 217. When rotary motor shaft 2121 turns in the clockside direction (looking from above), rotary motor coupler 2125 blade spins in the clockside direction and encounters the high side of the wall of cap coupler groove 217 and begin to rotate cap coupler 211 clockwise. As stepper motor 2110 lowers, the rotary motor coupler 2125 will engage the cap coupler groove 217, as shown in FIG. 20C. As shown in FIG. 20D, when stepper motor 2110 continues to lowers, the rotary motor 2120 and rotary motor shaft 2121 attached to rotary motor coupler 2125 will lower, pushing on cap coupler groove 217 and the cap coupler 211 will compress cap spring 213 against the bottom of cap sheath 212 and lower head 218. As shown in FIG. 20 E, head 218 can be lowered close to or in contact with the bottom of processing chamber 440, which can be a stator 354, and head 218 can be rotated to disrupt tissue. When stepper motor 2110 raises, rotary motor 2120 and rotary motor coupler 2125 raise up and cap spring 213 decompresses to push cap coupler 211 against rotary motor coupler 2125 to continue engagement.

In another embodiment of the Single Sample Singulator Instrument 2050, stepper motor 2110 controls the vertical position of rotary motor 2120 which is magnetically coupled to moveable disruptor 345 with a magnetic or paramagnetic element embedded with cap 210 as part of cap coupler 211 or as part of moveable disruptor 345 or head 218.

When rotary motor coupler 2125 is engaged with cap coupler 211 by mechanical coupling, magnetic coupling, pneumatic, or fluidic coupling, or other coupling methods, and rotary motor 2120 rotates, moveable disruptor 325 and head 218 are rotated. Stepper motor 2110 controls the vertical position of the rotary motor 2120 and thereby the vertical position of rotary motor coupler 2125, to raise or lower moveable disruptor 345 and head 218 in processing chamber 440. Combining rotation of rotary motor 2120 and movement of stepper motor 2110 enables many patterns of motion of moveable tissue disruptor 345 and head 218.

The inside walls of processing chamber 440 can be embodied in many different shapes. The inside walls of processing chamber 440 can be fluted to have sections with different depths. In a preferred embodiment, the inside wall can have a circular profile with the largest gap between the head 218 of moveable mechanical tissue disruptor 345 and the inside wall of preferably greater than or equal to 1 μm, or 2 μm, or 5 μm, or 10 μm, or 15 μm, or 20 μm, or 25 μm, or 30 μm, or 40 μm, or 50 μm, or 75 μm, or 100 μm, or 150 μm, or 200 μm, or 250 μm, or 500 μm, and 1000 μm or more, as well as any size in between.

Moveable tissue disruptor 345 can be embodied in many different shapes with many different profiles. In one embodiment, moveable tissue disruptor 345 can have a head 218 which is a rotor 353 with optional features, for example, grinding teeth 355 on the bottom of rotor 353 and grinding teeth 355 on stator 354 which is on the top surface of the bottom of the processing chamber 440 to assist in disruption of large pieces of tissue specimens 120 into smaller pieces or assist in the dissociation into single cells 1000 or nuclei 1050 or biomolecules 1070. As shown in FIGS. 8 and 18, the sides of head 218 can be a cylinder to create an inside gap 221 with the inside wall over the length of the cylinder. By raising and lowering head 218 without turning head 218, thereby using it as a moveable disruptor 345, the system can process specimen 101 by trituration. In another embodiment the sides of the head 218 can form a ball-like structure to create a gap with the inside wall in a small area and the bottom of processing chamber 440 can be rounded to match the ball-like structure to create a Dounce-like mechanical tissue disruptor 345. In other embodiments, multiple regions with gaps of the same or different sizes can be created by varying the side profile of moveable tissue disruptor 345 and the inner wall of processing chamber 440. In other embodiments, the stator can be stationary or movable (e.g., rotating). In other embodiments, the stator is non-porous. In other embodiments, either or both of the processing chamber or the rotor can have a circular or non-circular cross-section. In the case of a non-circular cross-section, the rotor is configured to rotate within the processing chamber. In its rotation, a portion of a wall of the rotor will have a gap between the rotor and the processing chamber of between about 150 and 250 micron.

Disruption of tissue can include a plurality of disruption steps, each involving positioning the head a different distance from floor of the chamber to produce gaps of different sizes. Typically, at each position, the head will rotate, further facilitating disruption or mixing. In certain embodiments, an organ can be auto-minced by the disrutor before tissue disruption into single cells 1000 or nuclei 1050 or other biological materials. Such a method can involve a first disruption step, which can include setting the head at a plurality of different distances from the floor of the chamber and rotating at each gap distance, to provide tissue with greater surface area and less distance for access by enzymes. A next step can involve incubating the auto-minced organ with enzymes or chemicals for tissue disruption into single cells 1000 or nuclei 1050. A next step can involve a second disruption step, which, in turn, can include setting the head at a plurality of different distances from the floor of the chamber and rotating the head.

Processes described here can be performed using one or more computer systems that can be networked together. Calculations can be performed in a cloud computing system in which data on the host computer is communicated through the communications network to a cloud computer that performs computations and that communicates, or outputs results to a user through a communications network. For example, nucleic acid sequencing can be performed on sequencing machines located at a user site. The resulting sequence data files can be transmitted to a cloud computing system where the sequence classification algorithm performs one or more operations of the methods described herein. At any step cloud computing system can transmit results of calculations back to the computer operated by the user.

Data can be transmitted electronically, e.g., over the Internet. Electronic communication can be, for example, over any communications network include, for example, a high-speed transmission network including, without limitation, Digital Subscriber Line (DSL), Cable Modem, Fiber, Wireless, Satellite and, Broadband over Powerlines (BPL). Information can be transmitted to a modem for transmission, e.g., wireless or wired transmission, to a computer such as a desktop computer. Alternatively, reports can be transmitted to a mobile device. Reports may be accessible through a subscription program in which a user accesses a website which displays the report. Reports can be transmitted to a user interface device accessible by the user. The user interface device could be, for example, a personal computer, a laptop, a smart phone or a wearable device, e.g., a watch, for example worn on the wrist.

### Example: Production of a Single Cell Suspension From Fresh Mouse Kidney

The Single Sample Singulator System 2000 can be operated in many configurations. As an example, an operator may wish to process a fresh mouse kidney specimen 101 into a single cell suspension 1000 and use reagents stored on Reagent Module 1430. The operator would remove cap 210 from cartridge 200 as shown in FIG. 8A and add a whole mouse kidney, or a part of mouse kidney, or part of a kidney that had been preminced to sample inlet port 425. The cap 210, which is a moveable disruptor, is replaced on processing chamber 440 with the bottom of cap sheath 212 seated on an annular seat in processing chamber 440. The now complete cartridge with a tissue specimen is placed on cartridge receiver tray 1510 and inserted into the Single Sample Singulator instrument 2050 with cartridge slide 1450. After the appropriate protocol is selected through user interface 740 on tablet 750, the Single Sample Singulator instrument 2050 heats thermal transfer plate to hold the processing chamber 440 at 37° C. and then begins processing kidney specimen 101.

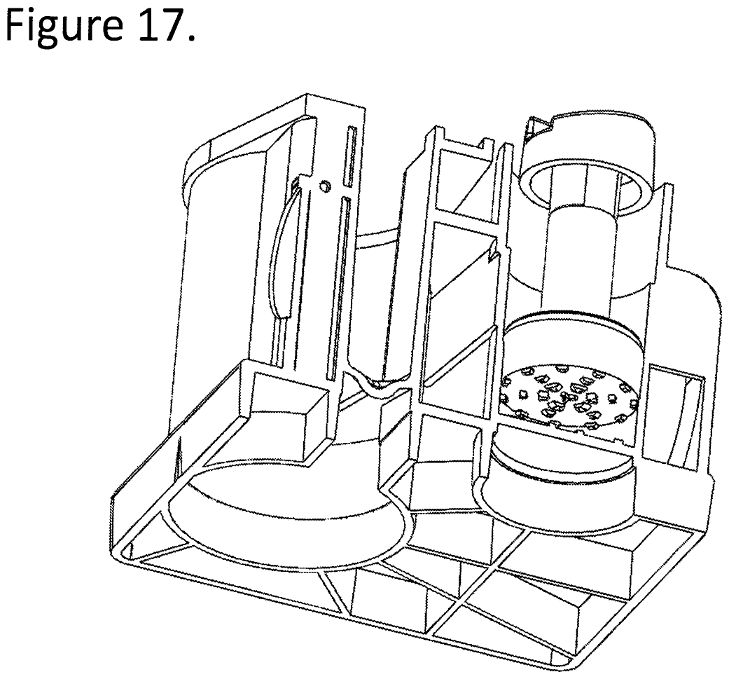

After initialization of electronic boards, the z-axis stepper motor 2110 moves the rotary motor 2120 down to engage rotary motor coupler 2125 with cap coupler 211. The control software 725 then selects the proper valve settings to pull two mL of mouse kidney reagent solution from Position 3 in temperature-controlled reagent storage chamber 1419 of reagent module 1430, as shown in FIG. 17, and deliver it through port 470 to processing chamber 440 where the mouse kidney has been placed.

If selected by the protocol, an auto-mince procedure to macerate the tissue is performed by the z-axis stepper motor 2110 moving rotary motor 2120, and therefore the mechanical tissue disruptor and head 218, which is functioning as a rotor 353, to 1.5 mm from the bottom of the processing chamber 440 and then rotor 353 is rotated clockwise for four seconds and then counterclockwise for four seconds at 95 rpm. Rotor 353 is lowered to 0.6 mm from the bottom and rotated clockwise for four seconds and counterclockwise for four seconds at 95 rpm. Rotor 353 is lowered to 0.3 mm from the bottom and rotated clockwise for four seconds and counterclockwise for four seconds at 95 rpm to complete the standard automince portion of the protocol.

For mouse kidney, the now auto-minced kidney specimen 101 is then incubated for twenty minutes with continuous top immersion mixing where rotor 353 is lowered into the top third of the mouse kidney reagent solution with kidney specimen 101 in preprocessor chamber 440 and the rotary motor 210 spins rotor 353 clockwise at 95 rpm in a continuous immersion mixing mode while the enzymatic formulation digests the extracellular matrix in the solid tissue to release cells.

After 20 min, the tissue is mechanically disrupted by lowering rotor 353 until it is 4.2 mm from the bottom, approximately 20% immersed into mouse kidney reagent solution with kidney specimen 101, and then the first mechanical disruption cycle is performed with rotor 353 rotating clockwise for four seconds and then rotating counterclockwise for four seconds at 95 rpm. The second disruption cycle is performed by lowering rotor 353 by 1.5 mm and rotating clockwise for four seconds and then counterclockwise for four seconds at 95 rpm. The third disruption cycle is by lowering rotor 353 by 0.9 mm and rotating clockwise for four seconds and counterclockwise for four seconds at 95 rpm. Then, the fourth and fifth disruptions cycles are performed with lowering rotor 353 by 0.6 mm each cycle with rotation clockwise for four seconds, counterclockwise for four seconds, then rotation clockwise for four seconds, and counterclockwise for four seconds at 95 rpm for each disruption cycle. For the sixth disruption cycle, the rotor 353 is raised 0.3 mm and then rotated clockwise for four seconds, counterclockwise for four seconds, clockwise for four seconds, and counterclockwise for four seconds at 95 rpm. For the seventh disruption cycle, the rotor 353 is lowered 0.6 mm and rotated clockwise for four seconds, counterclockwise for four seconds, rotated clockwise for four seconds, and counterclockwise for four seconds at 95 rpm. For the eight and final disruption cycle, the rotor 353 is lowered 0.3 mm in contact with the bottom surface of processing chamber 440 and rotated clockwise for four seconds, counterclockwise for four seconds, rotated clockwise for four seconds, and counterclockwise for four seconds at 95 rpm. Many other possible disruption profiles are enabled by this instant invention.

The mechanical tissue disruption occurs at two places: first, at the bottom of rotor 353 by grinding teeth 355 and the top of stator 354 with complementary grinding teeth 355 to mechanically dissociate the solid tissue in bottom gap 222 and secondly, the gap between the circumference of the rotor 353 and the inner wall of processing chamber 440 acts as an orifice to disrupt the tissue.

With the rotor 353 positioned at the bottom of processing chamber 440, syringe pump 2130 then pulls vacuum through the appropriate six way valve settings on vacuum trap port 467 to pull the dissociated mouse kidney single cell suspension through line 453, through 70 μm strainer 2711 where it drains down strain drain 451 and into output collector region 461 in processing chamber 460.

The control software 725 sets the selection of valve settings to pull two mL of HBSS-Ca—Mg from Position 13 in room temperature reagent storage chamber 1418 of reagent module 1430 as shown in FIG. 17 and deliver it through port 470 to processing chamber 440. Rotor 353 can be moved to mix any remaining dissociated cells with the HBSS-Ca-Mg and then with rotor 353 positioned at the bottom of processing chamber 440, syringe pump 2130 then pulls vacuum through the appropriate six way valve settings on vacuum trap port 467 to pull the HBSS-Ca—Mg and any remaining dissociated mouse kidney single cells suspension through line 453, through 70 μm strainer 2711, down strain drain 451 and into output collector region 461 in processing chamber 460. This process is then repeated to deliver and pull a second two mL of HBSS-Ca—Mg through processing chamber 440 and into processing chamber 460. The mouse kidney single cell 1000 suspension can then be pipetted out by opening processing chamber cap 465 and withdrawing the cell suspension from output collector region 461 using a pipettor.

The mouse kidney single cell 1000 suspension can be centrifuged at 300 g for five min to collect the cells as a pellet, the red blood cells lyzed for five min with a RBC lysis buffer, and the suspension centrifuged at 300 g for five min to collect the cells. As an example, a 262 mg of mouse kidney produced a single cell suspension 1000 by this process with a cell titer of 14,670,000 cells at a 85.5% viability as determined by counting on a Countess II with Trypan blue staining.

Other tissues or organs may benefit from different modes of mixing. The Single Sample Singulator System 2000 is designed to perform a plurality of mixing modalities. For example, top mixing is designed to position the bottom of head 218 at 15 mm above the bottom and rotate head 218 to mix the enzymatic or chemical dissolution solution 410 with the specimen 101. Shallow immersive mixing can be performed by continuously rotating head 218 as it is moved from 17.7 mm above the bottom down to 16.8 mm and back up again. Tritutation mixing can be performed by moving head 218 without rotation from 12.3 mm above the bottom down to 0.3 mm above the bottom. Many other mixing modalities are enabled.

### Example: Production a Single Nuclei Suspension From Flash Frozen Human Brain

The Single Sample Singulator System 2000 can be operated in many configurations to produce nuclei 1050 suspensions. As an example, an operator may wish to process a fresh mouse kidney specimen 101 into a single nuclei suspension 1050 and use reagents stored on Reagent Module 1430. The operator would remove cap 210 from cartridge 200 as shown in

FIG. 8 and add a whole mouse kidney, or a part of a kidney, or part of a kidney that had been preminced to sample inlet port 425. The cap 210, which is a tissue disruptor, is replaced on processing chamber 440 and the now complete cartridge with a tissue specimen 101 is placed on cartridge receiver tray 1510 and inserted into the Single Sample Singulator instrument 2050 with cartridge slide 1450. After the appropriate protocol is selected through user interface 740 on tablet 750, the Single Sample Singulator instrument 2050 cools thermal transfer plate 1470 to hold the processing chamber 440 and post-processing chamber 460 at 4° C. and then begins processing kidney specimen 101. The thermal transfer plate 1470 can also be preheated or precooled as needed.

After initialization of boards, the z-axis stepper motor 2110 moves the rotary motor 210 down to engage rotary motor coupler 2125 with cap coupler 211. The control software 725 then selects the valve settings to pull two mL of nuclei isolation solution 412 from Position 1 in temperature-controlled reagent storage chamber 1419 of reagent module 1430 as shown in FIG. 17 and deliver it through port 470 to post-processing chamber 440.

The tissue is then mechanically disrupted by lowering head 218 which will function as rotor 353 until it is 4.2 mm from the bottom, approximately 20% immersed into the nuclei isolation solution 412 with kidney specimen 101, and then the first mechanical disruption cycle is performed with moveable mechanical disruptor 345 and head 218 acting as a rotor 353 rotated clockwise for four seconds and then rotated counterclockwise for four seconds at 135 rpm. The second disruption cycle is by lowering rotor 353 by 1.5 mm and rotating clockwise for four seconds and then counterclockwise for four seconds at 135 rpm. The third disruption cycle is by lowering rotor 353 by 0.9 mm and rotating clockwise for four seconds and then rotating counterclockwise for four seconds at 135 rpm. Then, the fourth and fifth disruptions cycles are performed with lowering rotor 353 by 0.6 mm with rotation clockwise for four seconds, counterclockwise for four seconds, rotation clockwise for four seconds, and counterclockwise for four seconds at 135 rpm for each disruption cycle. For the sixth disruption cycle, the rotor 353 is raised 0.3 mm and then rotated clockwise for four seconds, counterclockwise for four seconds, clockwise for four seconds, and counterclockwise for four seconds at 135 rpm. For the seventh disruption cycle, the rotor 353 is lowered 0.6 mm and rotated clockwise for four seconds, counterclockwise for four seconds, rotated clockwise for four seconds, and counterclockwise for four seconds at 135 rpm. For the eight disruption cycle, the rotor 353 is lowered 0.3 mm and rotated clockwise for four seconds, counterclockwise for four seconds, rotated clockwise for four seconds, and counterclockwise for four seconds at 135 rpm.

The mechanical tissue disruption again occurs both at the bottom of rotor 353 by grinding teeth 355 and the top of stator 354 with complementary grinding teeth 355 mechanically dissociating the solid tissue in bottom gap 222 as well as any tissue passing between the circumference of the rotor 353 and the inner wall of processing chamber 440 in side gap 221.

With the rotor 353 positioned at the bottom of processing chamber 440, syringe pump 2130 then pulls vacuum through the appropriate six way valve settings on vacuum trap port 467 to pull the dissociated mouse kidney nuclei suspension through line 453, through a 40 μm strainer 2711 in post-processing chamber 460, down strain drain 451 and into output collector region 461.

The control software 725 sets the selection of valve settings to pull two mL of nuclei storage solution 413 from Position 2 in temperature-controlled reagent storage chamber 1419 of reagent module 1430 as shown in FIG. 17 and delivers it through port 470 to processing chamber 440. Rotor 353 can be moved to mix any remaining dissociated nuclei 1050 with the nuclei storage solution 413 and then with rotor 353 positioned at the bottom of processing chamber 440, syringe pump 2130 pulls vacuum through the appropriate six way valve settings on vacuum trap port 467 to pull the nuclei storage solution 413 and any remaining dissociated mouse kidney single nuclei 1050 suspension through line 453, through a 40 μm strainer 2711, down strain drain 451 and into output collector region 461. The mouse kidney single nuclei 1050 suspension can then be pipetted out by opening processing chamber cap 465 and withdrawing the cell suspension from output collector region 461.

The mouse kidney single cell 1050 suspension can be centrifuged at 500 g for 5 min to collect the cells as a pellet before resuspension in nuclei storage solution 413 or other media. As an example, a 108 mg mouse kidney produced by this process yielded a nuclei suspension 1050 with a titer of 24,225,000 as determined by counting on a Countess II with Trypan blue staining.

### Example: Processing Tissue Into Nuclei or Cells

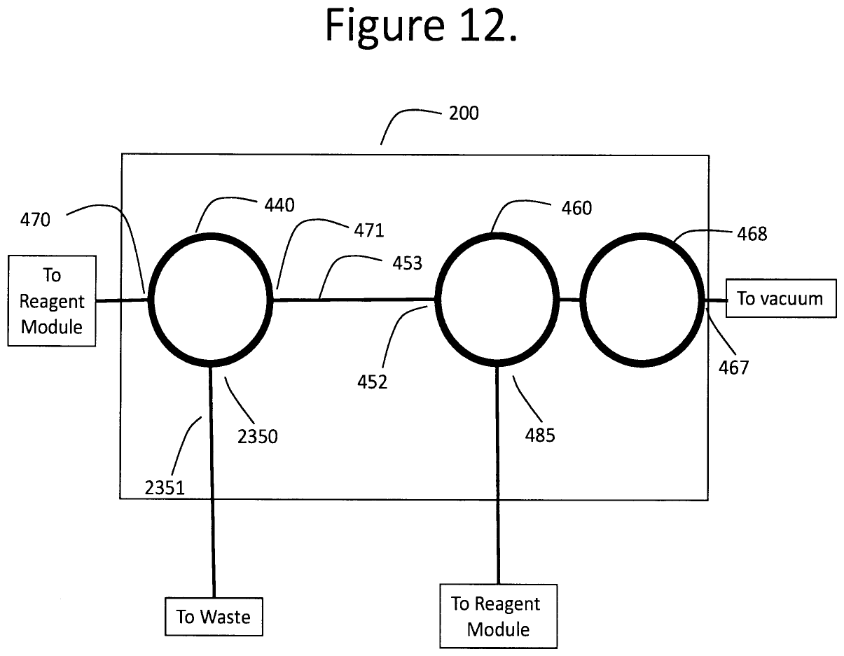

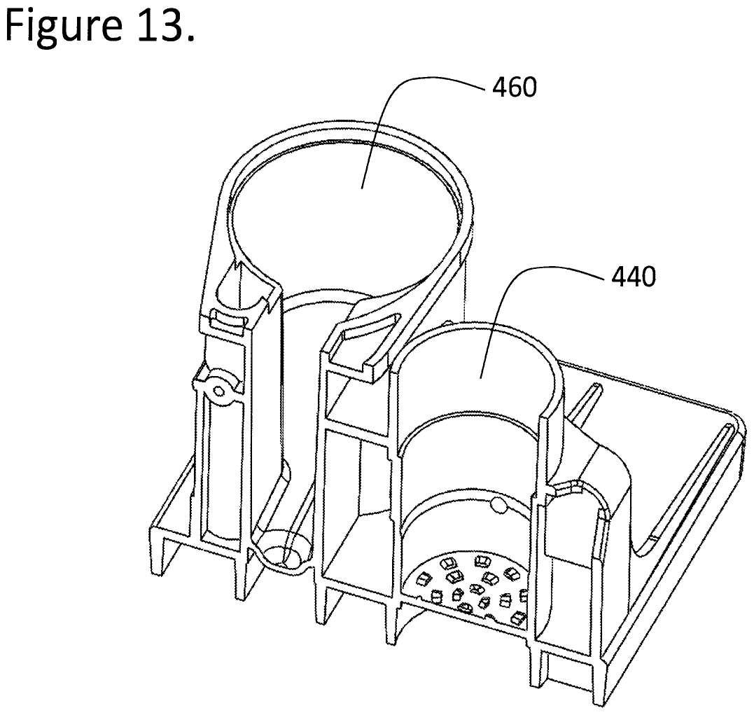

Referring to FIG. 12, a tissue specimen can be placed in the processing chamber 440 of cartridge 200 which has a reagent addition port 470 that connects to reagents in reagent block 415 as part of reagent module 1430; a waste port 2350 that connects waste line 2351 to waste; a port 471 connecting flexible tubing 453 to port 452 of post-processing chamber 460. Post-processing chamber 460 is in turn connected to reagents in reagent block 415 as part of reagent module 1430 through port 485 and also can contain or be connected to on cartridge vacuum trap 468 with port 467 which can connect to a vacuum source.

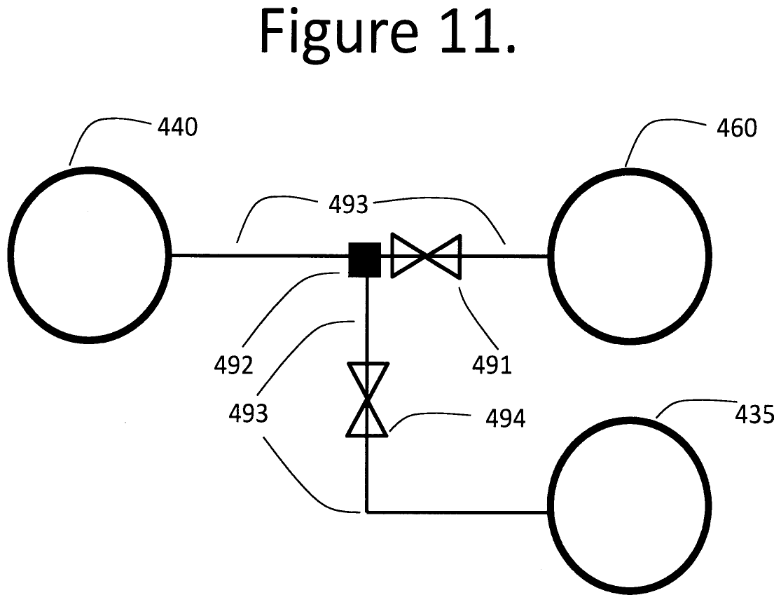

In one embodiment the waste is moved through additional ports or directed at valves, such as pinch valves 491, to on-cartridge waste 430. In one embodiment, illustrated in FIG. 11, processing chamber 440 is connected by flexible tubing 493 to three way junction 492 which is further connected by flexible tubing 493 to post-processing chamber 460 and cartridge waste chamber 435. Flow can be directed from processing chamber 440 to cartridge waste chamber 435 by closing pinch valve 491 while pinch valve 494 is open and applying vacuum to cartridge waste chamber 435. The cartridge waste chamber 435 can also be replaced by off cartridge waste as desired. Flow can be directed from processing chamber 440 to post-processing chamber 460 by opening pinch valve 491 while pinch valve 494 is closed and applying vacuum to post-processing chamber 460. Similar designs can be used to fluidically connect other devices such as flow cells, flow cytometry, nanodroplet single cell processors, sequencers, etc. as a device or module of the instrument.

In an embodiment, as shown in FIG. 8, a tissue specimen 150 is inserted into the processing chamber 440 of cartridge 200 through sample inlet port 425. The cap 210 is added and the cartridge 200 placed into an Tissue processing instrument 2010. In some embodiments cartridge 200 has a filter added in or over the channel leading to the waste port 2350 to prevent loss of the tissue to waste line 2351. In some embodiments, waste line 2351 has a pinch valve 491 to minimize the volume of liquid in the line. In other embodiments waste line 2351 has a T junction and one or more pinch valves 491 to direct the flow of liquid for example to an on-cartridge waste reservoir 430.

Selection of the appropriate cell or nuclei protocol for processing FFPE tissue specimens 150 and using the appropriate setup of reagent module 1430, as shown in FIGS. 8 and 9A, the instrument can add, for example, 2 mL of xylene from the reagent module 1430 to cartridge 200 through port 470 into processing chamber 440 containing FFPE tissue specimen 150. The xylene is then incubated for a time period selected from the range of 10 sec, 30 sec, 1 min, 5 min, 10 min, 15 min, 30 min or longer at room temperature. In some embodiments, as shown in FIG. 9B, rotor 218 is lowered into the xylene and rotated to circulate the xylene around the FFPE tissue specimen 150. In other embodiments the xylene is moved by changes of pressure applied through waste line 2351 through port 426 or liquid can be pumped in and out of waste line 2351 through port 426.

Referring to FIGS. 8 and 9C, vacuum is then applied to waste port 2355 and the xylene is then pulled from processing chamber 440 through waste channel 2350 and through waste line 2351 into the instrument.

The instrument can then perform rehydration, for example, by adding two mL of 100% ethanol from the reagent module 1430 to cartridge 200 and incubating for a time period selected from the range of 10 sec, 30 sec, 1 min, 5 min, 10 min, 15 min, 30 min or longer at room temperature or other temperature. The 100% ethanol is then removed through waste channel 2350 and the process repeated none, one, or more additional times with 100% ethanol. In some embodiments, 70% ethanol is then added from the reagent module 1430 to cartridge 200 and incubating for a time period selected from the range of 10 sec, 30 sec, 1 min, 5 min, 10 min, 15 min, 30 min or longer at room temperature or other temperature. The 70% ethanol is then removed through waste channel 2350 and the process repeated none, one, or more additional times with lower concentrations of ethanol, e.g., 50%, 30%, and 10%, before water or buffer such PBS is added.

The deparaffinized rehydrated FFPE tissue specimen 150 can have an optional crosslink reversal step. In one method, an enzymatic digestion is performed by adding up to two mL of proteinase K solution (0.005% proteinase K, 30 U/mg protein, in 50 mM Tris hydroxymethyl aminomethane hydrochloride (pH 7.0), 10 mM EDTA, and 10 mM sodium chloride), with optional DNase addition, and incubating for a time period selected from the range of 1 min, 5 min, 10 min, 15 min, 30 min, 60 min, 2 hours, 4 hours, 8 hours, 12 hours, 24 hours at 37° C. or up to 60° C. or other temperatures. The proteinase solution is then removed through waste channel 2350. Other methods such as heating the deparaffinized rehydrated FFPE can also be employed to reverse crosslinking.

The released nuclei 1050 suspension in nuclei storage buffer 413 in some embodiments can now pass through the pores in tissue ring 2300 or filter basket 2350 and is then pulled into the through fluidic line 453 through an optional filter(s) into post-processing chamber 460.

The processes can be in cartridges in, for example, two mL volumes. Sections of 5, 10, 20, 30, and 50 μm can be processed to optimize the thickness to recover intact nuclei. Dewaxing issues can be found with thicker slices. Incubation times, temperatures, and number of cycles for deparaffinization can be varied and xylene replacement formulations used (e.g., CitriSolv, HistoChoice, NeoClear, Ultraclear, Qiagen Deparaffinization Solution). For sample rehydration, successive ethanol incubations of decreasing concentration are needed. In addition to stepwise ethanol concentration reduction, the instrument fluidics can produce a continuous gradient between ethanol and other mixture components, to optimize the impact on the tissue, shorten the rehydration and other process times. The continuous gradient mode can improve the nuclei or cell morphology, yield, and RNA quality compared to the standard stepwise gradients.

Single nuclei sequencing can be performed by methods comprised of SMARTSeq and nanodrop snRNA-Seq or with the 10x Genomics Chromium Single Cell Gene Expression Flex. SMARTSeq exhibits more uniform transcriptome coverage with lower numbers of nuclei. For SMARTSeq, individual nuclei can be isolated, transferred into individual wells of a microtiter plate on ice, and cDNA prepared using the SMART-Seq® Single Cell Kit (Takara Bio). The yield and quality of nuclei suspensions can be tested using qPCR on the ACTB gene. The snRNA-Seq approach can use nanodrops to encapsulate the nuclei and perform library preparation. The amount and quality of the cDNA can be measured by qPCR of ACTB and by electrophoresis to determine if the cDNA has an appropriate size range, is free of contaminating small fragments, and present in sufficient yield for Nextera (Illumina) or other library preparation. Sequence data metrics include percent of uniquely mapped reads, mapping rates of reads (exonic, intronic, intergenic), read coverage uniformity, mtDNA contamination, cell type-specific marker genes to measure cell type diversity of the resulting single nuclei populations, and principal component and hierarchical clustering analyses.

### Example: Processing Mouse Kidney and Lung Tissue Into Nuclei

Mouse lung or kidney samples were harvested under an IACUC approved protocol. Tissue was rinsed in PBS and a piece cut off and weighed. The tissue was then place in a Singulator 100 sample cartridge and the tissue dissociated into nuclei using the Singulator 100 Small Volume protocol and the S2 Genomics Nuclei Isolation Reagent (NIR) and Nuclei Storage Reagent (NSR) reagents. The resultant suspension of nuclei was removed from the cartridge and centrifuged for 5 minutes at 500 g and 4° C. The resulting pellet was resuspended in 1 ml of NSR and the nuclei yield was measured on a Nexcelom K2 automated cell counter with AOPI fluorescent staining.

### Example: Processing FFPE Human Brain Samples Into Nuclei and Preparing a Single Nuclei Sequencing Library

A block of human brain preserved in FFPE was sectioned into slices. Two 50 μm slices and one 100 μm slice of the FFPE specimen 150 were then added through sample inlet port 425 to the processing chamber 440 of a cartridge 200, the grinder assembly 345 placed onto processing chamber 440 and the cartridge now containing the FFPE specimens 150 loaded into a Singulator instrument 100.

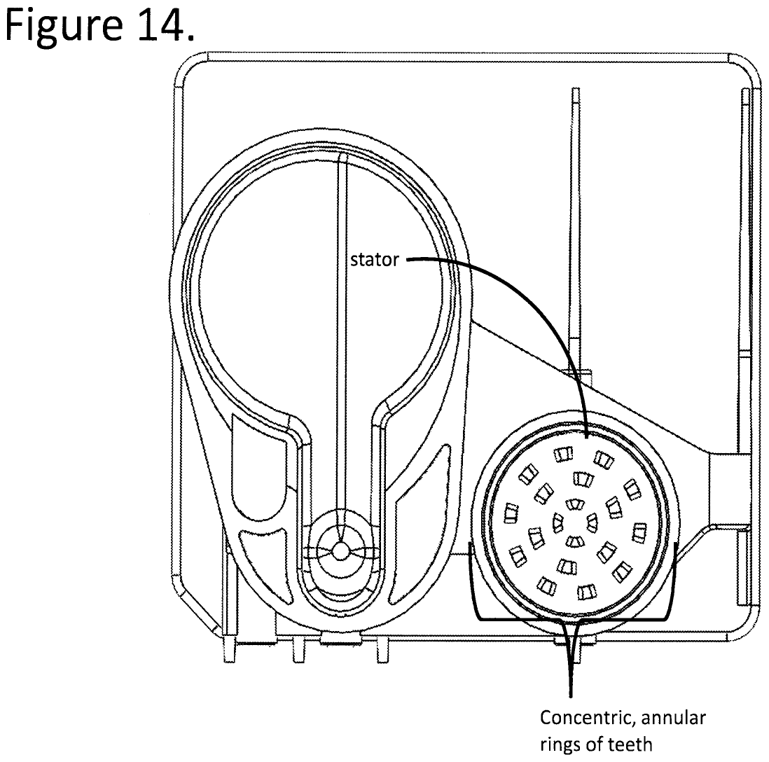

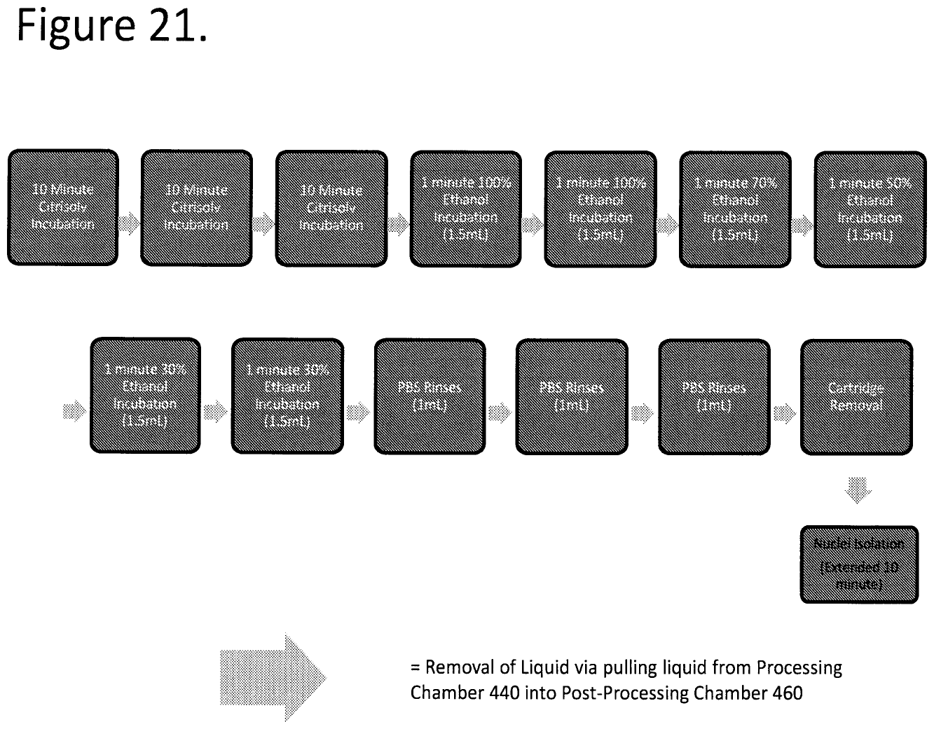

The instrument had been programmed to perform the steps as shown in FIG. 21 with the reagents added to the cartridge 200 sequentially through reagent addition port 470 for the appropriate incubation time with grinder assembly 345 lowered to cover port 471 to prevent the FFPE specimens 150 from being removed with waste, and then, after incubation, the reagent pulled by vacuum on vacuum trap port 467 into post-processing chamber 460 leaving the FFPE specimen 150 in processing chamber 440. Following the third PBS rinse as shown in FIG. 21, the cartridge 200 was removed from instrument 100, the grinder assembly 345 withdrawn and the now deparaffinized and rehydrated FFPE specimen 150 slices placed into a new cartridge 200 with grinder assembly 345 as shown in FIGS. 14 and 15. The deparaffinized and rehydrated FFPE specimen 150 slices were then processed into nuclei as described above using the Singulator 100 Small Volume protocol and the NIR and NSR reagents producing an ‘automated processed FFPE sample’ 160. In parallel, the same procedure of FIG. 21 was carried out manually in tubes with no automation until dissociation into nuclei using cartridge 200 on instrument 100 producing a ‘manually processed FFPE sample 165.

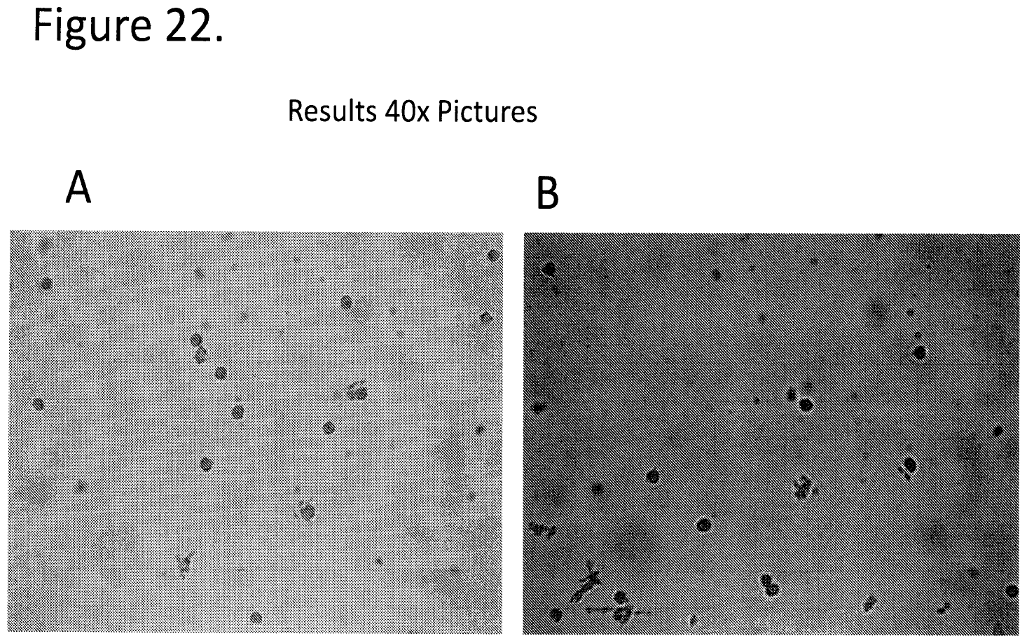

The manually processed FFPE sample 165 produced 12,940,000 nuclei and the automated processed FFPE sample 160 produced 9,840,000 nuclei of similar appearance as shown in FIGS. 22A and B respectively. The method also can produce at least two million nuclei from a tissue sample (e.g., brain) of 11.25 mm³.

The manually processed FFPE sample 165 and the automated processed FFPE sample 160 were then processed into single nuclei libraries using the 10x Genomics Chromium Single Cell Gene Expression Flex kit and protocol as described by the manufacturer. Nuclei were pelleted and resuspended in 500 μl of 1X Fix and Perm Buffer for 1 hr at 20° C., rinsed in 0.5x PBS with 0.02% BSA twice with 850 g spins for 5 min, and then resuspended in 500 μL of quench buffer, supplemented to 10% glycerol with 0.1× Enhancer and frozen overnight at −80° C. The Cell Gene Expression Flex Kit was run as described by 10x Genomics protocol targeting recovery of 8,000 nuclei and PCR amplified for 15 cycles. The resulting single nuclei libraries from FFPE specimens were then analyzed on an Agilent TapeStation for size and concentration.

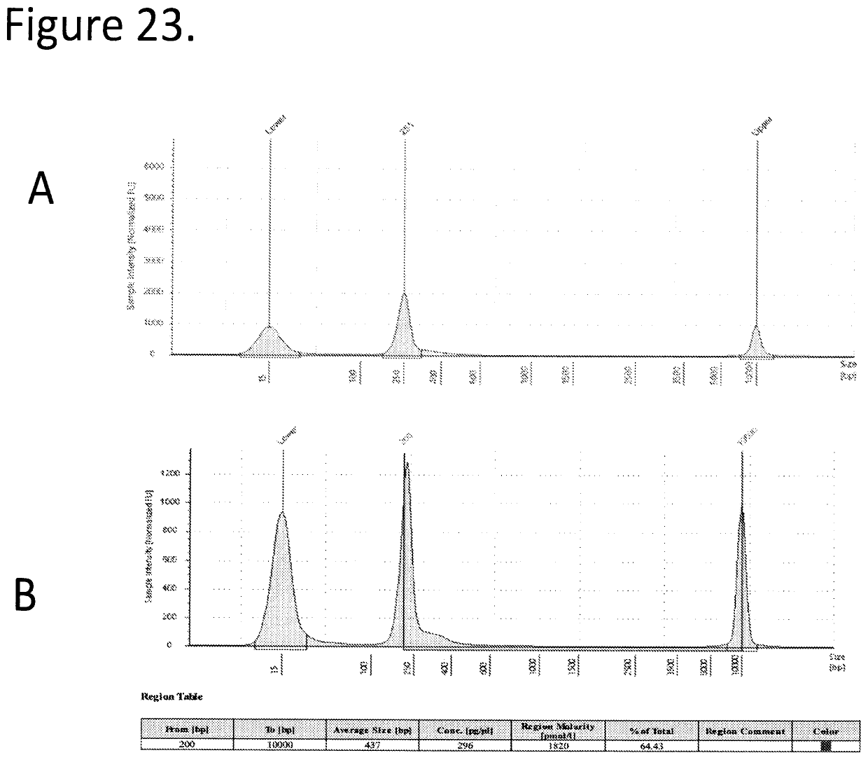

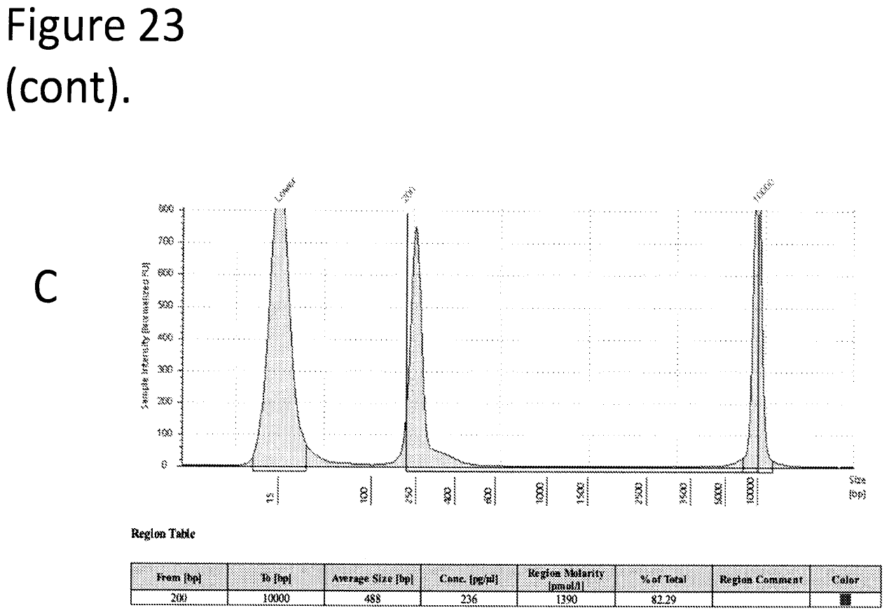

FIG. 23A shows a representative trace from the manufacturer for the kit and this was not generated in this work. FIG. 23B shows the library from the manually processed FFPE sample 165 which was dissociated into nuclei using the cartridge 200 with a peak at the expected location and a concentration of 296 pg/μl. FIG. 23C shows the library from the automated processed FFPE sample 160 with a peak at the expected location and a concentration of 236 pg/μl. The data in FIG. 23 demonstrate the production of single nuclei RNA sequencing libraries using a fully automated deparapfinnization and rehydration process followed by automated nuclei production on the instrument 100 using cartridges 200 and by a manual deparaffinnization and rehydration process followed by automated nuclei production on the instrument.

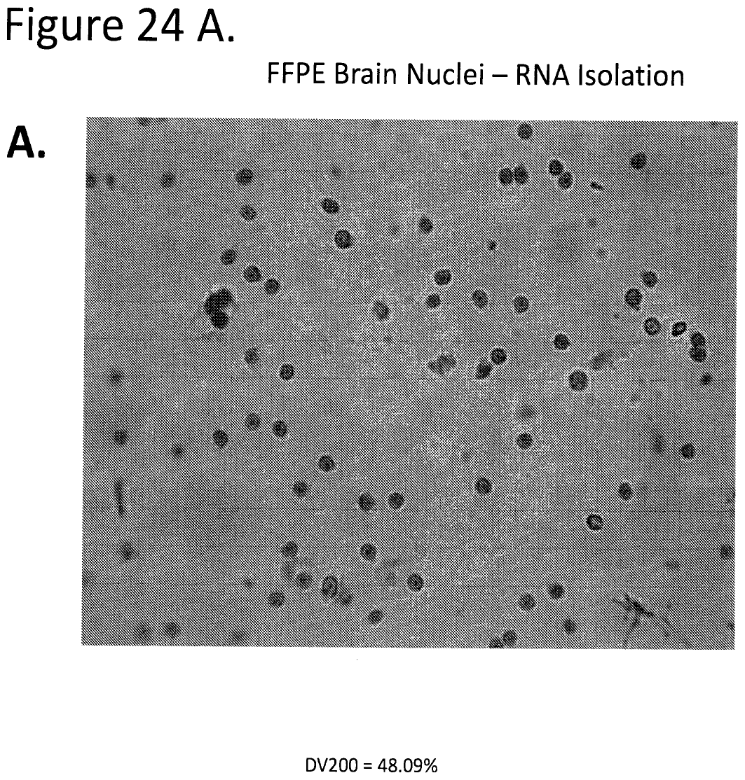

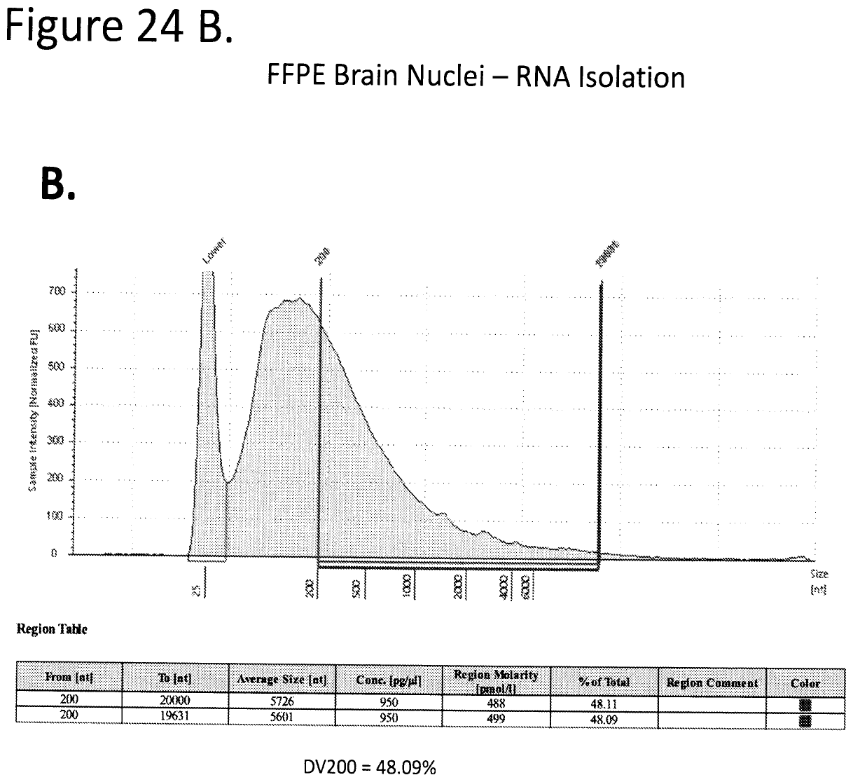

In another experiment, the automated procedure (deparaffinization, rehydration, and nuclei production) using the Singulator instrument 100 was performed as described above and in FIG. 21 with two 80 μm FFPE 150 brain slices and approximately 7 million nuclei recovered, FIG. 24A. The nuclei were then processed using the RNA isolation from ˜5 million nuclei using the Qiagen FFPE Kit starting at the Proteinase K Digestion Step and analyzed on the Tapestation. The result shown in FIG. 24B was recovery of RNA with a DV200 score of 48.09%, consistent with degradation of RNA length commonly found with FFPE processed samples.

Nucleic acids isolated from cells or nuclei isolated from FFPE tissue using methods and systems herein can be prepared into nucleic acid libraries. Library preparation can comprise, for RNA, isolation of RNA, such as mRNA, from the sample; and reverse transcription of RNA into cDNA. DNA can be isolated from a sample and fragmented. Double-stranded nucleic acids, can be blunt-ended or provided with a single nucleotide overhang. Sequencing adapters can be attached to these molecules. Adapters can be attached by blunt end ligation or sticky in ligation, e.g., were the nucleic acid molecules are provided with a single nucleotide overhang. Sequencing adapters can comprise, for example, a sequencing primer binding site, a sample barcode, a molecular barcode, and a cell-flow sequence. Such molecules can then be amplified by, for example, PCR.

### EXEMPLARY EMBODIMENTS

### Exemplary Embodiments

1. A cartridge for dissociating tissue, comprising:
a processing chamber comprising a stator, a side wall, a top orifice, and a first processing chamber port positioned in the side wall; and
a grinder assembly comprising a plunger comprising a rotor, the grinder assembly slidably positioned in the processing chamber through the top orifice; wherein:
   the stator comprises a plurality of teeth arranged in a spaced-apart array of rings; and
   the rotor comprises one or more central teeth and a plurality of teeth arranged in a spaced-apart array of rings, wherein one ring of teeth is positioned at or substantially at a circumference of the rotor;
   wherein the rings in the stator and the rings in the rotor are positioned such that when the rotor contacts the stator, rings of teeth in the stator mesh with the one or more central teeth and rings of teeth in the rotor.
2. The cartridge of embodiment 1, further comprising a post-processing chamber comprising a port that fluidically communicates with the first processing chamber port, wherein the post-processing chamber comprises a single-cell suspension or single-nuclei suspension.
3. The cartridge of embodiment 1, wherein a plurality of the teeth have a trapezoidal cross-section.
4. The cartridge of embodiment 1, wherein the stator and the rotor each comprise three rings of teeth.
5. The cartridge of embodiment 1, wherein the rotor comprises an inner ring comprising six teeth, a middle ring comprising six teeth and an outer ring comprising 11 teeth.
6. The cartridge of embodiment 1, wherein the rotor comprises teeth at a density of about 1 tooth per 0.0025 mm² to about 1 tooth per 0.10 mm², e.g., about 1 tooth per 0.05 mm².
7. The cartridge of embodiment 1, wherein the stator comprises an inner ring comprising four teeth, a middle ring comprising six teeth and an outer ring comprising 10 teeth.
8. The cartridge of embodiment 1, wherein the stator comprises teeth at a density of about 1 tooth per 0.002 mm² to about 1 tooth per 0.08 mm², e.g., about 1 tooth per 0.04 mm².
9. The cartridge of embodiment 1, wherein one or more teeth have a height of about 500 microns and a width of about 1 mm to 2 mm.
10. The cartridge of embodiment 1, wherein the processing chamber has a volume between 5 mL and 100 mL, e.g., between 10 mL and 50 mL, e.g. between 10 mL and 20 mL.
11. The cartridge of embodiment 1, wherein the processing chamber has a cross-sectional area of between about 78 mm² (e.g., radius of about 5 mm) and about 1256 mm² (e.g., radius of about 20 mm), e.g., about 452 mm² (e.g., radius of about 12 mm).
12. The cartridge of embodiment 1, comprising a tissue sample no greater than 20 mg, no greater than 10 mg, no greater than 5 mg, no greater than 2 mg, or no greater than 1 mg.
13. The cartridge of embodiment 1, comprising a gap between the rotor and the sidewall of about 1 micron and 500 microns.
14. The cartridge of embodiment 1, wherein the first processing chamber port is positioned above a top of the rotor when the rotor is fully depressed.
15. The cartridge of embodiment 1, wherein the grinder assembly further comprises a cap attached to the plunger and configured to cover the orifice and position the grinder assembly in the processing chamber.
16. The cartridge of embodiment 15, wherein the plunger is spring-biased toward the cap.
17. The cartridge of embodiment 15, wherein the cap comprises a key slot to engage an actuator.
18. The cartridge of embodiment 1, further comprising one or a plurality of:
a strain chamber comprising a strainer having pores no greater than about 40 microns (e.g., no greater than about 20 microns), and an optional second strainer having pores at least about 40 microns and no greater than about 200 microns; wherein the strain chamber communicates with the processing chamber through the second processing port;
a waste port that communicates with the third processing chamber port;
a post-processing chamber comprising: a first post-processing chamber port that communicates with the strain chamber; and a second post-processing chamber port; and a third post-processing chamber port; and
a vacuum trap comprising: a first vacuum trap port that communicates with the post-processing chamber through the second post-processing chamber port; and a second vacuum trap chamber port.
19. The cartridge of embodiment 18, wherein the processing chamber and the post-processing chamber communicate through a fluidic channel.
20. The cartridge of embodiment 18, wherein the third processing chamber port and the waste port communicate through a fluidic channel.
21. The cartridge of embodiment 18, wherein the first strainer has pores no more than about 40 microns (e.g., no greater than about 20 microns) and the second strainer has pores between about 140 microns to about 200 microns.
22. The cartridge of embodiment 18, wherein the first strainer has pores about 145 microns. the second strainer has pores about 40 microns and a third filter has pores of about 20 microns.
23. The cartridge of embodiment 18, wherein the second processing port communicates with the post-processing chamber through a port in a cap of the post-processing chamber.
24. The cartridge of embodiment 18, wherein the rotor of the plunger is biased toward the cap (e.g., spring biased).
25. The cartridge of embodiment 18, wherein the rotor has sufficient clearance from the processing chamber walls to allow liquid, cells and nuclei to pass around the rotor during depression, and the first processing port is positioned above the rotor when fully depressed.
26. The cartridge of embodiment 18, wherein the strain container is configured as an assembly comprising a basket and a lid, wherein the basket has an open top that is closed by the lid, the lid is attached to the plunger, wherein the assembly fits into the processing chamber, and wherein moving the plunger up and down along the Z axis moves the basket up and down through the solution.
27. The cartridge of embodiment 18, wherein the second processing port is covered by a filter, e.g., a dual filter, having pores too small for cells and/or nuclei to pass.
28. The cartridge of embodiment 18, wherein the second processing port communicates with the post-processing chamber through a port in a cap of the post-processing chamber.
29. The cartridge of embodiment 18, wherein processing chamber, the post-processing chamber and the waste chamber communicate through fluidic channels that meet at a three-way junction and have one or more switchable valves.
30. The cartridge of embodiment 18, comprising a valve between the processing chamber and the post-processing chamber and between the vacuum chamber and either or both of the processing chamber and the post-processing chamber.
31. The cartridge of embodiment 18, further comprising a detection window.
32. The cartridge of embodiment 18, further comprising a waste chamber comprising a first waste chamber port that communicates with the processing chamber.
33. A cartridge comprising a rotor with an array of teeth including a center tooth, and complementary stator and outlet port connected to an instrument with a compartment containing a single-cell or single-nuclei suspension.
34. A system comprising:
(a) an instrument comprising:
   (i) a cartridge interface configured to engage a cartridge;
   (ii) a fluidic subsystem comprising:
      (1) one or more fluid lines connecting the one or more containers with one or more fluid ports in the cartridge interface; and
      (2) one or more pumps configured to apply positive or negative pressure to one or more fluid ports and to move liquids and/or gasses into and/or out of the one or more fluid ports
      (3) an optional waste chamber communicating with a pump;
   (iii) a physical dissociation subsystem comprising an actuator, a linear driver (e.g., a stepper motor or a pneumatic driver) that drives an actuator in an up-down (Z axis) direction, and a rotary motor that rotates the actuator around the Z axis; and
   (v) a control subsystem comprising a digital computer comprising a processor and memory, wherein the memory comprises code that, when executed by the processor, instructs the system to perform one or more operations;
(b) an enzymatic and chemical dissociation subsystem, which may be positioned inside or outside of the instrument, comprising:
   (1) a reagent module comprising one or more containers containing one or more liquids and/or gasses and/or solids; and
(c) a cartridge of embodiment 1 or embodiment 18, releasably engaged with the cartridge interface, wherein:
   (A) the first processing port is engaged with a first interface port in the cartridge interface that is connected with a pump that delivers reagents from the reagent module to the first cartridge port;
   (B) the rotor assembly is engaged with the actuator;
   (C) the waste port is engaged with a second interface port in the cartridge interface that is connected with a pump that positive or negative pressure to the waste port;
   (D) the third post-processing chamber port is engaged with a third interface port in the cartridge interface that is connected with a pump that delivers reagents from the reagent module to the third post-processing port;
   (E) the second vacuum trap port is engaged with a fourth interface port in the cartridge interface that is connected with a pump that positive or negative pressure to the waste port;
   wherein the operations comprise introducing fluids from the reagent module into the processing chamber, introducing fluids from the reagent module into the post-processing chamber; stepping and/or rotating the rotor assembly, moving liquid from the processing chamber through the cartridge waste port; and moving a suspension from the processing chamber to the post-processing chamber.
35. The system of embodiment 34, wherein the interface ports comprise fittings that engage the cartridge ports (e.g., nozzles, pogo pins, a flared connectors).
36. The system of embodiment 34, wherein the control subsystem comprises a user interface configured to accept input from a user in the execution of the instructions.
37. The system of embodiment 34, wherein the instrument further comprises one or more of:
(vi) a magnetic post-processing module comprising a source of magnetic force, wherein the magnetic force is positioned to form a magnetic field in the post-processing chamber;
(vii) a measurement subsystem that performs optical imaging to measure titer, clumping, and/or viability of cells or nuclei or other characteristics of the sample in the cartridge; and
(viii) a temperature control subsystem comprising a heating and/or cooling element positioned to heat and/or cool the processing chamber and/or the post-processing chamber.
38. The system of embodiment 37, wherein the measurement subsystem is configured to measure, at one or more time points, characteristics of a sample in the post-processing chamber.
39. The system of embodiment 38, wherein the characteristic is selected from viability or degree of cell or nuclei dissociation or cell type or cell surface markers.
40. The system of embodiment 38, wherein the characteristic is selected from degree of deparaffinization or rehydration.
41. The system of embodiment 37, wherein the temperature control subsystem comprises a thermal transfer plate and a temperature controller, e.g., a Peltier, a strip resistive heater, one or more circulating fluids.
42. The system of embodiment 34, wherein the containers contain one or more of: a deparaffinizing solution, a cross-link reversal solution, one or more rehydrating solutions, protease solutions, a buffer comprising a detergent, a lysis buffer, a resuspension buffer, dissociation solution, nuclei isolation solution, and nuclei storage solution.
43. The system of embodiment 42, wherein the deparaffinizing solution comprises a compound that dissolves paraffin, e.g., xylene or a xylene substitute such as Citrisolv, Everclear™ Xylene substitute, Histoclear, etc.
44. The system of embodiment 42, wherein the rehydrating solutions are selected from H2O and aqueous solutions of ethanol of different concentrations.
45. The system of embodiment 42, wherein the protease solutions comprise one or more of proteinase K, a collagenase (e.g., collagenases type I, II, III, IV, and others), elastase, trypsin, papain, hyaluronidase, chymotrypsin, neutral protease, clostripain, caseinase, and neutral protease (Dispase®).
46. The system of embodiment 42, wherein the lysis buffer comprises an aqueous buffer and a detergent.
47. The system of embodiment 42, wherein the resuspension buffer comprises an aqueous buffer, and a compound for maintaining osmolarity compatible with cells and/or nuclei, e.g., bovine serum albumin.
48. The system of embodiment 42, wherein the dissociation solution comprises one or more enzymes that cleave extracellular matrix.
49. The system of embodiment 42, wherein the cross-link reversal solution comprises an enzyme or chemical that cleaves formalin cross-links, e.g., Proteinase K or IHC retrieval reagent.
50. The system of embodiment 42, wherein the nuclei isolation solution comprises a buffer compatible with nuclei.
51. The system of embodiment 42, wherein the nuclei storage solution comprises an aqueous buffer, a salt, and Ca++ and/or Mg++.
52. The system of embodiment 34, wherein one of the pumps provides vacuum to a fluid port engaging the second vacuum trap port.
53. The system of embodiment 34, wherein the actuator engages the rotor assembly through a drive fitting, e.g., slot, cross, phillips, polygon, or interlocking teeth.
54. The system of embodiment 34, further comprising a barcode reader.
55. The system of embodiment 34, further comprising:
(c) an analysis subsystem, wherein an input port of the analysis module communicates with the post-processing chamber.
56. The system of embodiment 55, wherein the analysis system communicates with the post-processing chamber through a fluidic channel or fluid handling robot.
57. The system of embodiment 55, wherein the analysis module performs an analysis selected from one or more of: DNA sequencing, next generation DNA sequencing, next generation DNA sequencing, proteomic analysis, genomic analysis, gene expression analysis, gene mapping, carbohydrate characterization and profiling, lipid characterization and profiling, flow cytometry, imaging, DNA or RNA microarray analysis, metabolic profiling, enzymatic assays, functional analysis, and mass spectrometry.
58. A method comprising producing single cells or organelles from no more than 20 mg tissue with at least 70% of produced cells intact.
59. The method of embodiment 58, comprising:
providing no more than 20 micrograms of tissue in a processing chamber of the cartridge of embodiment 1;
grinding the tissue in the processing chamber with the teeth between the rotor and the stator to produce a suspension of single cells or organelles;
moving the suspension out of the processing chamber through the first processing chamber port.
60. The method of embodiment 59, further comprising:
removing the suspension of biological material from the processing chamber.
61. The method of embodiment 59, wherein the processing chamber further comprises one or more enzymes for digesting extracellular matrix.
62. The method of embodiment 59, wherein the processing chamber further comprises one or more detergents for lysing cell membranes.
63. The method of embodiment 59, wherein the processing chamber further comprises liquid having a viscosity that slows the rate of degradation of RNA or other biomolecules during or after tissue disruption.
64. The method of embodiment 59, wherein disrupting comprises positioning a disruption surface of the head a defined distance from a bottom surface of the processing chamber and rotating the head to disrupt tissue in the processing chamber.
65. The method of embodiment 59, wherein disrupting comprises positioning a disruption surface of the head with respect to a bottom surface of the processing chamber at a plurality of different gap distances and, at each gap distance, rotating the head.
66. The method of embodiment 65, wherein at least one gap distance, at least some portion of the disruption head contacts some portion of the bottom surface.
67. The method of embodiment 65, wherein the widest gap distance between a flat portion of the surface and flat portion of the bottom of the chamber is no more than any of 6 mm, 5 mm 4 mm, 3 mm, 2 mm, 1 mm, 500 um, 250 um, 100 um, 75 um, 50 um, 25 um, 20 um, 15 um, 10 um, 5 um, 4 um, 3 um, 2 um, or 1 um.
68. The method of embodiment 65, wherein the plurality of gap distances between a flat portion of the grinding surface and flat portion of the bottom of the chamber is any of 2, 3, 4, 5, 6, 7, 8, 9 or 10 and the largest gap distance is no more than any of 6 mm, 5 mm 4 mm, 3 mm, 2 mm, 1 mm, 500 um, 250 um, 100 um, 75 um, 50 um, 25 um, 20 um, 15 um, 10 um, 5 um, 4 um, 3 um, 2 um, or 1 um.
69. The method of embodiment 65, comprising:
disrupting tissue with the tissue disruptor;
incubating the disrupted tissue with at least one enzyme that digests extracellular matrix; and
disrupting the incubated tissue with the tissue disruptor.
70. The method of embodiment 59, wherein the fluidic subsystem applies a vacuum to a cartridge port communicating with the processing chamber to move the suspension of biological material.
71. The method of embodiment 59, wherein the cartridge further comprises a strainer and the suspension of biological material entering the processing chamber is strained to remove particulate matter.
72. The method of embodiment 59, further comprising, after moving the suspension of biological material, using the fluidics subsystem to introduce a liquid into the processing chamber through a cartridge port and then using the fluidics subsystem to move the liquid into the processing chamber.
73. The method of embodiment 59, further comprising, using the fluidics subsystem to introduce one or more liquids comprising one or more reagents through a cartridge port into the processing chamber.
74. The method of embodiment 73, wherein the reagent comprises an enzyme or a particle comprising a binding agent (e.g., a binding agent directed against a target on a cell surface or a surface of a nucleus, virus or other biological target).
75. The method of embodiment 59, wherein the tissue comprises a target cell and the method further comprises:
contacting the suspension of biological material in the processing chamber with solid particles comprising binding agents that bind to the target cells and sequester bound target cells within the suspension of biological material.
76. The method of embodiment 75, further comprising separating the bound target cells from the suspension.
77. The system of embodiment 42, wherein the single nuclei produced from FFPE are processed into a single nuclei library and sequenced.
78. A method comprising deparaffinizing and rehydrating an FFPE tissue sample; disrupting the deparaffinized and rehydrated FFPE tissue sample using a system and cartridge as described herein (e.g., a system of claim 34); isolating cells and/or nuclei individually or in groups from the disrupted sample; and generating an adapter-tagged nucleic acid library from nucleic acid from the isolated cells and/or nuclei.
79. The method of embodiment 78, wherein generating the adapter-tagged library comprises hybridizing left hand side and right hand side nucleic acid probes to sequences in mRNA molecules in the isolated cells and/or nuclei, ligating the probes, and performing primer extension of the ligated probe with a primer comprising a recognition sequence and a barcode.
80. The method of embodiment 78, wherein generating the adapter-tagged library comprises isolating nucleic acids from the isolated cells and/or nuclei, and attaching adapter molecules.
81. The method of embodiment 78, wherein the tissue sample is deparaffinized and rehydrated in a system and cartridge as described herein.
82. The method of embodiment 78, wherein the nucleic acid is DNA or RNA, e.g., mRNA.
83. The method of embodiment 78, further comprising sequencing the nucleic acid library, e.g., by DNA cluster sequencing.
84. A method comprising using a cartridge as described herein (e.g., a cartridge of claim 1) to produce at least two million nuclei from an FFPE tissue sample.
85. The method of embodiment 84, comprising producing at least 9 million nuclei or at least 12 million nuclei or from an FFPE tissue sample.
86. The method of embodiment 84, comprising producing at least two million nuclei from a tissue sample of no more than 5 mm³.
87. The method of embodiment 84, comprising producing at least two million nuclei from a tissue sample of no more than 10 mm³.
88. The method of embodiment 84, comprising producing at least two million nuclei from a tissue sample of no more than 11.25 mm³.
89. The method of embodiment 84, comprising producing at least two million nuclei from a tissue sample of no more than 15 mm³.
90. The method of embodiment 84, comprising producing at least two million nuclei from a tissue sample of no more than 20 mm³.
91. The method of claim 84, comprising producing the nuclei using a system as disclosed herein, e.g., a system of claim 34.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The term “or” is, unless indicated otherwise, non-exclusive, i.e., encompassing both “and” and “or.”

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present). Both plural and singular means may be included. The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

All patents, patent applications, published applications, treatises and other publications referred to herein, both supra and infra, are incorporated by reference in their entirety. If a definition and/or description is set forth herein that is contrary to or otherwise inconsistent with any definition set forth in the patents, patent applications, published applications, and other publications that are herein incorporated by reference, the definition and/or description set forth herein prevails over the definition that is incorporated by reference.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

What is claimed is:

1. A cartridge for dissociating tissue, comprising:
a processing chamber comprising a stator, a side wall, a top orifice, and a first processing chamber port positioned in the side wall; and
a grinder assembly comprising a plunger comprising a rotor, the grinder assembly slidably positioned in the processing chamber through the top orifice; wherein:
the stator comprises a plurality of teeth arranged in a spaced-apart array of rings; and
the rotor comprises one or more central teeth and a plurality of teeth arranged in a spaced-apart array of rings, wherein one ring of teeth is positioned at or substantially at a circumference of the rotor;
wherein the rings in the stator and the rings in the rotor are positioned such that when the rotor contacts the stator, rings of teeth in the stator mesh with the one or more central teeth and rings of teeth in the rotor.

2. The cartridge of claim 1, further comprising a post-processing chamber comprising a port that fluidically communicates with the first processing chamber port, wherein the post-processing chamber comprises a single-cell suspension or single-nuclei suspension.

3. The cartridge of claim 1, wherein a plurality of the teeth have a trapezoidal cross-section.

4. The cartridge of claim 1, wherein the stator and the rotor each comprise three rings of teeth.

5. The cartridge of claim 1, wherein the rotor comprises an inner ring comprising six teeth, a middle ring comprising six teeth and an outer ring comprising 11 teeth.

6. The cartridge of claim 1, wherein the rotor comprises teeth at a density of about 1 tooth per 0.0025 mm² to about 1 tooth per 0.10 mm², e.g., about 1 tooth per 0.05 mm².

7. The cartridge of claim 1, wherein the stator comprises an inner ring comprising four teeth, a middle ring comprising six teeth and an outer ring comprising 10 teeth.

8. The cartridge of claim 1, wherein the stator comprises teeth at a density of about 1 tooth per 0.002 mm² to about 1 tooth per 0.08 mm², e.g., about 1 tooth per 0.04 mm².

9. The cartridge of claim 1, wherein one or more teeth have a height of about 500 microns and a width of about 1 mm to 2 mm.

10. The cartridge of claim 1, wherein the processing chamber has a volume between 5 mL and 100 mL, e.g., between 10 mL and 50 mL, e.g. between 10 mL and 20 mL.

11. The cartridge of claim 1, wherein the processing chamber has a cross-sectional area of between about 78 mm² (e.g., radius of about 5 mm) and about 1256 mm² (e.g., radius of about 20 mm), e.g., about 452 mm² (e.g., radius of about 12 mm).

12. The cartridge of claim 1, comprising a tissue sample no greater than 20 mg, no greater than 10 mg, no greater than 5 mg, no greater than 2 mg, or no greater than 1 mg.

13. The cartridge of claim 1, comprising a gap between the rotor and the sidewall of about 1 micron and 500 microns.

14. The cartridge of claim 1, wherein the first processing chamber port is positioned above a top of the rotor when the rotor is fully depressed.

15. The cartridge of claim 1, wherein the grinder assembly further comprises a cap attached to the plunger and configured to cover the orifice and position the grinder assembly in the processing chamber.

16. The cartridge of claim 15, wherein the plunger is spring-biased toward the cap.

17. The cartridge of claim 15, wherein the cap comprises a key slot to engage an actuator.

18. The cartridge of claim 1, further comprising one or a plurality of:
a strain chamber comprising a strainer having pores no greater than about 40 microns (e.g., no greater than about 20 microns), and an optional second strainer having pores at least about 40 microns and no greater than about 200 microns; wherein the strain chamber communicates with the processing chamber through the second processing port;
a waste port that communicates with the third processing chamber port;
a post-processing chamber comprising: a first post-processing chamber port that communicates with the strain chamber; and a second post-processing chamber port; and a third post-processing chamber port; and
a vacuum trap comprising: a first vacuum trap port that communicates with the post-processing chamber through the second post-processing chamber port; and a second vacuum trap chamber port.

19. The cartridge of claim 18, wherein the processing chamber and the post-processing chamber communicate through a fluidic channel.

20. The cartridge of claim 18, wherein the third processing chamber port and the waste port communicate through a fluidic channel.

21. The cartridge of claim 18, wherein the first strainer has pores no more than about 40 microns (e.g., no greater than about 20 microns) and the second strainer has pores between about 140 microns to about 200 microns.

22. The cartridge of claim 18, wherein the first strainer has pores about 145 microns, the second strainer has pores about 40 microns and a third filter has pores of about 20 microns.

23. The cartridge of claim 18, wherein the second processing port communicates with the post-processing chamber through a port in a cap of the post-processing chamber.

24. The cartridge of claim 18, wherein the rotor of the plunger is biased toward the cap (e.g., spring biased).

25. The cartridge of claim 18, wherein the rotor has sufficient clearance from the processing chamber walls to allow liquid, cells and nuclei to pass around the rotor during depression, and the first processing port is positioned above the rotor when fully depressed.

26. The cartridge of claim 18, wherein the strain container is configured as an assembly comprising a basket and a lid, wherein the basket has an open top that is closed by the lid, the lid is attached to the plunger, wherein the assembly fits into the processing chamber, and wherein moving the plunger up and down along the Z axis moves the basket up and down through the solution.

27. The cartridge of claim 18, wherein the second processing port is covered by a filter, e.g., a dual filter, having pores too small for cells and/or nuclei to pass.

28. The cartridge of claim 18, wherein the second processing port communicates with the post-processing chamber through a port in a cap of the post-processing chamber.

29. The cartridge of claim 18, wherein processing chamber, the post-processing chamber and the waste chamber communicate through fluidic channels that meet at a three-way junction and have one or more switchable valves.

30. The cartridge of claim 18, comprising a valve between the processing chamber and the post-processing chamber and between the vacuum chamber and either or both of the processing chamber and the post-processing chamber.

31. The cartridge of claim 18, further comprising a detection window.

32. The cartridge of claim 18, further comprising a waste chamber comprising a first waste chamber port that communicates with the processing chamber.

33. A cartridge comprising a rotor with an array of teeth including a center tooth, and complementary stator and outlet port connected to an instrument with a compartment containing a single-cell or single-nuclei suspension.

34. A system comprising:
(a) an instrument comprising:
(i) a cartridge interface configured to engage a cartridge;
(ii) a fluidic subsystem comprising:
(1) one or more fluid lines connecting the one or more containers with one or more fluid ports in the cartridge interface; and
(2) one or more pumps configured to apply positive or negative pressure to one or more fluid ports and to move liquids and/or gasses into and/or out of the one or more fluid ports
(3) an optional waste chamber communicating with a pump;
(iii) a physical dissociation subsystem comprising an actuator, a linear driver (e.g., a stepper motor or a pneumatic driver) that drives an actuator in an up-down (Z axis) direction, and a rotary motor that rotates the actuator around the Z axis; and
(iv) a control subsystem comprising a digital computer comprising a processor and memory, wherein the memory comprises code that, when executed by the processor, instructs the system to perform one or more operations;
(b) an enzymatic and chemical dissociation subsystem, which may be positioned inside or outside of the instrument, comprising:
(1) a reagent module comprising one or more containers containing one or more liquids and/or gasses and/or solids; and
(c) a cartridge of claim 1 or claim 18, releasably engaged with the cartridge interface, wherein:
(A) the first processing port is engaged with a first interface port in the cartridge interface that is connected with a pump that delivers reagents from the reagent module to the first cartridge port;
(B) the rotor assembly is engaged with the actuator;
(C) the waste port is engaged with a second interface port in the cartridge interface that is connected with a pump that positive or negative pressure to the waste port;
(D) the third post-processing chamber port is engaged with a third interface port in the cartridge interface that is connected with a pump that delivers reagents from the reagent module to the third post-processing port;
(E) the second vacuum trap port is engaged with a fourth interface port in the cartridge interface that is connected with a pump that positive or negative pressure to the waste port;
wherein the operations comprise introducing fluids from the reagent module into the processing chamber, introducing fluids from the reagent module into the post-processing chamber; stepping and/or rotating the rotor assembly, moving liquid from the processing chamber through the cartridge waste port; and moving a suspension from the processing chamber to the post-processing chamber.

35. The system of claim 34, wherein the interface ports comprise fittings that engage the cartridge ports (e.g., nozzles, pogo pins, a flared connectors).

36. The system of claim 34, wherein the control subsystem comprises a user interface configured to accept input from a user in the execution of the instructions.

37. The system of claim 34, wherein the instrument further comprises one or more of:
(v) a magnetic post-processing module comprising a source of magnetic force, wherein the magnetic force is positioned to form a magnetic field in the post-processing chamber;
(vi) a measurement subsystem that performs optical imaging to measure titer, clumping, and/or viability of cells or nuclei or other characteristics of the sample in the cartridge; and
(vii) a temperature control subsystem comprising a heating and/or cooling element positioned to heat and/or cool the processing chamber and/or the post-processing chamber.

38. The system of claim 37, wherein the measurement subsystem is configured to measure, at one or more time points, characteristics of a sample in the post-processing chamber.

39. The system of claim 38, wherein the characteristic is selected from viability or degree of cell or nuclei dissociation or cell type or cell surface markers.

40. The system of claim 38, wherein the characteristic is selected from degree of deparaffinization or rehydration.

41. The system of claim 37, wherein the temperature control subsystem comprises a thermal transfer plate and a temperature controller, e.g., a Peltier, a strip resistive heater, one or more circulating fluids.

42. The system of claim 34, wherein the containers contain one or more of: a deparaffinizing solution, a cross-link reversal solution, one or more rehydrating solutions, protease solutions, a buffer comprising a detergent, a lysis buffer, a resuspension buffer, dissociation solution, nuclei isolation solution, and nuclei storage solution.

43. The system of claim 42, wherein the deparaffinizing solution comprises a compound that dissolves paraffin, e.g., xylene or a xylene substitute such as Citrisolv, Everclear™ Xylene substitute, Histoclear, etc.

44. The system of claim 42, wherein the rehydrating solutions are selected from H₂O and aqueous solutions of ethanol of different concentrations.

45. The system of claim 42, wherein the protease solutions comprise one or more of proteinase K, a collagenase (e.g., collagenases type I, II, III, IV, and others), elastase, trypsin, papain, hyaluronidase, chymotrypsin, neutral protease, clostripain, caseinase, and neutral protease (Dispase®),

46. The system of claim 42, wherein the lysis buffer comprises an aqueous buffer and a detergent.

47. The system of claim 42, wherein the resuspension buffer comprises an aqueous buffer, and a compound for maintaining osmolarity compatible with cells and/or nuclei, e.g., bovine serum albumin.

48. The system of claim 42, wherein the dissociation solution comprises one or more enzymes that cleave extracellular matrix.

49. The system of claim 42, wherein the cross-link reversal solution comprises an enzyme or chemical that cleaves formalin cross-links, e.g., Proteinase K or IHC retrieval reagent.

50. The system of claim 42, wherein the nuclei isolation solution comprises a buffer compatible with nuclei.

51. The system of claim 42, wherein the nuclei storage solution comprises an aqueous buffer, a salt, and Ca⁺⁺ and/or Mg⁺⁺.

52. The system of claim 34, wherein one of the pumps provides vacuum to a fluid port engaging the second vacuum trap port.

53. The system of claim 34, wherein the actuator engages the rotor assembly through a drive fitting, e.g., slot, cross, phillips, polygon, or interlocking teeth.

54. The system of claim 34, further comprising a barcode reader.

55. The system of claim 34, further comprising:
(c) an analysis subsystem, wherein an input port of the analysis module communicates with the post-processing chamber.

56. The system of claim 55, wherein the analysis system communicates with the post- processing chamber through a fluidic channel or fluid handling robot.

57. The system of claim 55, wherein the analysis module performs an analysis selected from one or more of: DNA sequencing, next generation DNA sequencing, next generation DNA sequencing, proteomic analysis, genomic analysis, gene expression analysis, gene mapping, carbohydrate characterization and profiling, lipid characterization and profiling, flow cytometry, imaging, DNA or RNA microarray analysis, metabolic profiling, enzymatic assays, functional analysis, and mass spectrometry.

58. A method comprising producing single cells or organelles from no more than 20 mg tissue with at least 70% of produced cells intact.

59. The method of claim 58, comprising:
providing no more than 20 micrograms of tissue in a processing chamber of the cartridge of claim 1;
grinding the tissue in the processing chamber with the teeth between the rotor and the stator to produce a suspension of single cells or organelles;
moving the suspension out of the processing chamber through the first processing chamber port.

60. The method of claim 59, further comprising:
removing the suspension of biological material from the processing chamber.

61. The method of claim 59, wherein the processing chamber further comprises one or more enzymes for digesting extracellular matrix.

62. The method of claim 59, wherein the processing chamber further comprises one or more detergents for lysing cell membranes.

63. The method of claim 59, wherein the processing chamber further comprises liquid having a viscosity that slows the rate of degradation of RNA or other biomolecules during or after tissue disruption.

64. The method of claim 59, wherein disrupting comprises positioning a disruption surface of the head a defined distance from a bottom surface of the processing chamber and rotating the head to disrupt tissue in the processing chamber.

65. The method of claim 59, wherein disrupting comprises positioning a disruption surface of the head with respect to a bottom surface of the processing chamber at a plurality of different gap distances and, at each gap distance, rotating the head.

66. The method of claim 65, wherein at least one gap distance, at least some portion of the disruption head contacts some portion of the bottom surface.

67. The method of claim 65, wherein the widest gap distance between a flat portion of the surface and flat portion of the bottom of the chamber is no more than any of 6 mm, 5 mm 4 mm, 3 mm, 2 mm, 1 mm, 500 um, 250 um, 100 um, 75 um, 50 um, 25 um, 20 um, 15 um, 10 um, 5 um, 4 um, 3 um, 2 um, or 1 um.

68. The method of claim 65, wherein the plurality of gap distances between a flat portion of the grinding surface and flat portion of the bottom of the chamber is any of 2, 3, 4, 5, 6, 7, 8, 9 or 10 and the largest gap distance is no more than any of 6 mm, 5 mm 4 mm, 3 mm, 2 mm, 1 mm, 500 um, 250 um, 100 um, 75 um, 50 um, 25 um, 20 um, 15 um, 10 um, 5 um, 4 um, 3 um, 2 um, or 1 um.

69. The method of claim 65, comprising:
disrupting tissue with the tissue disruptor;
incubating the disrupted tissue with at least one enzyme that digests extracellular matrix; and
disrupting the incubated tissue with the tissue disruptor.

70. The method of claim 59, wherein the fluidic subsystem applies a vacuum to a cartridge port communicating with the processing chamber to move the suspension of biological material.

71. The method of claim 59, wherein the cartridge further comprises a strainer and the suspension of biological material entering the processing chamber is strained to remove particulate matter.

72. The method of claim 59, further comprising, after moving the suspension of biological material, using the fluidics subsystem to introduce a liquid into the processing chamber through a cartridge port and then using the fluidics subsystem to move the liquid into the processing chamber.

73. The method of claim 59, further comprising, using the fluidics subsystem to introduce one or more liquids comprising one or more reagents through a cartridge port into the processing chamber.

74. The method of claim 73, wherein the reagent comprises an enzyme or a particle comprising a binding agent (e.g., a binding agent directed against a target on a cell surface or a surface of a nucleus, virus or other biological target).

75. The method of claim 59, wherein the tissue comprises a target cell and the method further comprises:
contacting the suspension of biological material in the processing chamber with solid particles comprising binding agents that bind to the target cells and sequester bound target cells within the suspension of biological material.

76. The method of claim 75, further comprising separating the bound target cells from the suspension.

77. The system of claim 42, wherein the single nuclei produced from FFPE are processed into a single nuclei library and sequenced.

78. A method comprising:
deparaffinizing and rehydrating an FFPE tissue sample;
disrupting the deparaffinized and rehydrated FFPE tissue sample using a system and cartridge as described herein (e.g., a system of claim 34);
isolating cells and/or nuclei individually or in groups from the disrupted sample; and
generating an adapter-tagged nucleic acid library from nucleic acid from the isolated cells and/or nuclei.

79. The method of embodiment 78, wherein generating the adapter-tagged library comprises hybridizing left hand side and right hand side nucleic acid probes to sequences in mRNA molecules in the isolated cells and/or nuclei, ligating the probes, and performing primer extension of the ligated probe with a primer comprising a recognition sequence and a barcode.

80. The method of embodiment 78, wherein generating the adapter-tagged library comprises isolating nucleic acids from the isolated cells and/or nuclei, and attaching adapter molecules.

81. The method of embodiment 78, wherein the tissue sample is deparaffinized and rehydrated in a system and cartridge as described herein.

82. The method of embodiment 78, wherein the nucleic acid is DNA or RNA, e.g., mRNA.

83. The method of embodiment 78, further comprising sequencing the nucleic acid library, e.g., by DNA cluster sequencing.

84. A method comprising using a cartridge as disclosed herein (e.g., a cartridge of claim 1) to produce at least two million nuclei from an FFPE tissue sample.

85. The method of claim 84, comprising producing at least 9 million nuclei or at least 12 million nuclei or from an FFPE tissue sample.

86. The method of claim 84, comprising producing at least two million nuclei from a tissue sample of no more than 5 mm³.

87. The method of claim 84, comprising producing at least two million nuclei from a tissue sample of no more than 10 mm³.

88. The method of claim 84, comprising producing at least two million nuclei from a tissue sample of no more than 11.25 mm³.

89. The method of claim 84, comprising producing at least two million nuclei from a tissue sample of no more than 15 mm³.

90. The method of claim 84, comprising producing at least two million nuclei from a tissue sample of no more than 20 mm³.

91. The method of claim 84, comprising producing the nuclei using a system as disclosed herein, e.g., a system of claim 34.
